SEQUENCE LISTING (84 sequences)

SEQ ID NO: 1 - DNA, 15 bp, Artificial Sequence
  Active core domain of the DNA-enzyme developed by Santoro and Joyce
ggctagctacaacga

SEQ ID NO: 2 - DNA, 15 bp, Artificial Sequence
  misc_feature (1) ... (15): Consensus sequence of a DNA-enzyme according to the invention
  The nucleotide in positions 8 need not be present
  n is a or g or c or t
ggmtmghndnnnmgd

SEQ ID NO: 3 - DNA, 29 bp, Artificial sequence
  Specific base sequences of Sections I and III separated by a core domain.  Section I: positions 1 to 7.  Section III: positions 23 to 29
  misc_feature (8)..(22): The illustrated core domain represents a preferred core domain embodiment
  misc_feature (8)..(22): see Sequence No. 2
  n is a or g or c or t
gtcatgaggmtmghndnnnmgdggttagg

SEQ ID NO: 4 - DNA, 31 bp, Artificial sequence
  Specific base sequences of Sections I and III separated by a core domain. Section I: positions 1 to 8.  Section III: positions 23 to 31
  misc_feature (9)..(23): The illustrated core domain represents a preferred core domain embodiment
  misc_feature (9)..(23): See Sequence No. 2
  n is a or g or c or t
tgtcatgaggmtmghndnnnmgdggttaggg

SEQ ID NO: 5 - DNA, 33 bp, Artificial sequence
  Specific base sequences of Sections I and III separated by a core domain. Section 1: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
atgtcatgaggmtmghndnnnmgdggttagggg

SEQ ID NO: 6 - DNA, 29 bp, Artificial Sequence
  Specific base sequences of Sections I and III separated by a core domain. Section I: positions 1 to 7.  Section III: positions 23 to 29
  misc_feature (8)..(22): The illustrated core domain represents a preferred core domain embodiment
  misc_feature (8)..(22): See Sequence No. 2
  n is a or g or c or t
gtcgtggggmtmghndnnnmgdgattagg

SEQ ID NO: 7 - DNA, 30 bp, Artificial Sequence
  Specific base sequence of Sections I and III separated by core domain.  Section I: positions 1 to 8, Section III: positions 24 to 30
  misc_feature (9)..(23): The illustrated core domain represents a preferred core domain embodiment
  misc_feature (9)..(23): See Sequence No. 2
  n is a or g or c or t
tgtcgtggggmtmghndnnnmgdgattagg

SEQ ID NO: 8 - DNA, 31 bp, Artificial sequence
  Specific base sequences of Sections I and III separated by a core domain.  Section I: positions 1 to 9, Section III: positions 25 to 31
  misc_feature (10)..(24): The illustrated core domain represents a preferred core domain embodiment
  misc_feature (10)..(24): See Sequence No. 2
  n is a or g or c or t
atgtcgtggggmtmghndnnnmgdgattagg

SEQ ID NO: 9 - DNA, 29 bp, Artificial sequence
  Specific base sequences of Sections I and III separated by a core domain.  Section I: positions 1 to 7, Section III: positions 23 to 29
  misc_feature (8)..(22): The illustrated core domain represents a preferred core domain embodiment
  misc_feature (8)..(22): See Sequence NO. 2
  n is a or g or c or t
ttgttgaggmtmghndnnnmgdggtctca

SEQ ID NO: 10 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
cttgttgaggmtmghndnnnmgdggtctcac

SEQ ID NO: 11 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
tcttgttgaggmtmghndnnnmgdggtctcacc

SEQ ID NO: 12 - DNA, 29 bp, Artificial sequence
  Specific base sequences of Sections I and III separated by a core domain. Section I: position 1 to 7, Section III: positions 23 to 29
  misc_feature (8)..(22): The illustrated core domain represents a preferred core domain embodiment
  misc_feature (8)..(22): See Sequence No. 2
  n is a or g or c or t
ttgttgaggmtmghndnnnmgdagtctca

SEQ ID NO: 13 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
cttgttgaggmtmghndnnnmgdagtctcan

SEQ ID NO: 14 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
tcttgttgaggmtmghndnnnmgdagtctcann

SEQ ID NO: 15 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
ggcctgaggmtmghndnnnmgdctcaggg

SEQ ID NO: 16 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
cggcctgaggmtmghndnnnmgdctcaggga

SEQ ID NO: 17 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
tcggcctgaggmtmghndnnnmgdctcagggag

SEQ ID NO: 18 - DNA, 29 bp, Artificial sequence
  Specific base sequences of Sectons I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
tgcttgaggmtmghndnnnmgdcgcaggg

SEQ ID NO: 19 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
ctgcttgaggmtmghndnnnmgdcgcagggn

SEQ ID NO: 20 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
tctgcttgaggmtmghndnnnmgdcgcagggnn

SEQ ID NO: 21 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
gtgtggaggmtmghndnnnmgdtccatag

SEQ ID NO: 22 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
ggtgtggaggmtmghndnnnmgdtccatagg

SEQ ID NO: 23 - DNA, 33 bp, Artificial sequence
  Specific base sequences of Sections I and III separated by a core domain. Section I: positions 1 to 9, Section III: positions 25 to 33
  misc_difference (10)..(24): The illustrated core domain represents a preferred core domain embodiment
  misc_feature (10)..(24): See Sequence No. 2
  n is a or g or c or t
tggtgtggaggmtmghndnnnmgdtccataggc

SEQ ID NO: 24 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
acgtggaggmtmghndnnnmgdtcagacg

SEQ ID NO: 25 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
gacgtggaggmtmghndnnnmgdtcagacgn

SEQ ID NO: 26 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
cgacgtggaggmtmghndnnnmgdtcagacgnn

SEQ ID NO: 27 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
gtggggaggmtmghndnnnmgdtcagact

SEQ ID NO: 28 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
ggtggggaggmtmghndnnnmgdtcagactc

SEQ ID NO: 29 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
gggtggggaggmtmghndnnnmgdtcagactcc

SEQ ID NO: 30 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
gtgggtcggmtmghndnnnmgdgcagcag

SEQ ID NO: 31 - DNA, 30 bp, Artificial sequence
  Specific base sequences of Sections I and III separated by a core domain. Section I: positions 1 to 8, Section III: positions 24 to 30
  misc_feature (9)..(23): The illustrated core domain represents a preferred core domain embodiment
  misc_feature (9)..(23): See Sequence No. 2
  n is a or g or c or t
agtgggtcggmtmghndnnnmgdgcagcag

SEQ ID NO: 32 - DNA, 31 bp, Artificial sequence
  Specific base sequences of Sections I and III separated by a core domain. Section I: positions 1 to 9, Section III: positions 25 to 31
  misc_feature (10)..(24): The illustrated core domain represents a preferred core domain embodiment
  misc_feature (10)..(24): See Sequence No. 2
  n is a or g or c or t
gagtgggtcggmtmghndnnnmgdgcagcag

SEQ ID NO: 33 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
cgcttgaggmtmghndnnnmgdaaatctg

SEQ ID NO: 34 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
gcgcttgaggmtmghndnnnmgdaaatctgt

SEQ ID NO: 35 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
tgcgcttgaggmtmghndnnnmgdaaatctgtc

SEQ ID NO: 36 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
cgcttgaggmtmghndnnnmgdgaatctg

SEQ ID NO: 37 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
gcgcttgaggmtmghndnnnmgdgaatctgn

SEQ ID NO: 38 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
tgcgcttgaggmtmghndnnnmgdgaatctgnn

SEQ ID NO: 39 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
ctccagaggmtmghndnnnmgdatgtgga

SEQ ID NO: 40 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
gctccagaggmtmghndnnnmgdatgtggaa

SEQ ID NO: 41 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
agctccagaggmtmghndnnnmgdatgtggaat

SEQ ID NO: 42 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
ctccaggggmtmghndnnnmgdaggtgga

SEQ ID NO: 43 - DNA, 30 bp, Artificial sequence
  Specific base sequences of Sections I and III separated by a core domain. Section I: positions 1 to 8, Section III: positions 24 to 30
  misc_feature (9)..(23): The illustrated core domain represents a preferred core domain embodiment
  misc_feature (9)..(23): See Sequence No. 2
  n is a or g or c or t
gctccaggggmtmghndnnnmgdaggtgga

SEQ ID NO: 44 - DNA, 31 bp, Artificial sequence
  Specific base sequences of Sections I and III separated by a core domain. Section I: positions 1 to 9, Section III: positions 25 to 31
  misc_feature (10)..(24): The illustrated core domain represents a preferred core domain embodiment
  misc_feature (10)..(24): See Sequence No. 2
  n is a or g or c or t
agctccaggggmtmghndnnnmgdaggtgga

SEQ ID NO: 45 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
ggtacgaggmtmghndnnnmgdtcctggt

SEQ ID NO: 46 - DNA, 31 bp, Artificial sequence
  Specific base sequences of Sections I and III separated by a core domain. Section I: positions 1 to 8, Section III: positions 24 to 31
  misc_feature (9)..(23): The illustrated core domain represents a preferred core domain embodiment
  misc_feature (9)..(23): See Sequence No. 2
  n is a or g or c or t
gggtacgaggmtmghndnnnmgdtcctggta

SEQ ID NO: 47 - DNA, 33 bp, Artificial sequence
  Specific base sequences of Sections I and III separated by a core domain. Section I: positions 1 to 9, Section III: positions 25 to 33
  misc_feature (10)..(24): The illustrated core domain represents a preferred core domain embodiment
  misc_feature (10)..(24): See Sequence No. 2
  n is a or g or c or t
cgggtacgaggmtmghndnnnmgdtcctggtag

SEQ ID NO: 48 - DNA, 29 bp, Artificial sequence
  Specific base sequences of Sections I and III separated by a core domain. Section I: positions 1 to 7, Section III: positions 23 to 29
  misc_feature (8)..(22): The illustrated core domain represents a preferred core domain embodiment
  misc_feature (8)..(22): See Sequence No. 2
  n is a or g or c or t
ggtgcggggmtmghndnnnmgdtcttggc

SEQ ID NO: 49 - DNA, 30 bp, Artificial sequence
  Specific base sequences of Sections I and III separated by a core domain. Section I: positions 1 to 8, Section III: positions 24 to 30
  misc_feature (9)..(23): The illustrated core domain represents a preferred core domain embodiment
  misc_feature (9)..(23): See Sequence No. 2
  n is a or g or c or t
gggtgcggggmtmghndnnnmgdtcttggc

SEQ ID NO: 50 - DNA, 31 bp, Artificial sequence
  Specific base sequences of Sections I and III separated by a core domain. Section I: positions 1 to 9, Section III: positions 25 to 31
  misc_feature (10)..(24): The illustrated core domain represents a preferred core domain embodiment
  misc_feature (10)..(24): See Sequence No. 2
  n is a or g or c or t
cgggtgcggggmtmghndnnnmgdtcttggc

SEQ ID NO: 51 - DNA, 28 bp, Artificial sequence
  Sequence targeted against human Rhinovirus 14. Section I: positions 1 to 6, Section III: positions 22 to 28
  misc_feature (7)..(21): The illustrated core domain represents a preferred core domain embodiment
  misc_feature (7)..(21): See Sequence No. 2
  n is a or g or c or t
gtgggaggmtmghndnnnmgdtttaagg

SEQ ID NO: 52 - DNA, 30 bp, Artificial sequence
  Sequence targeted against human Rhinovirus 14. Section I: positions 1 to 7, Section III: positions 23 to 30
  misc_feature (8)..(22): The illustrated core domain represents a preferred core domain embodiment
  misc_feature (8)..(22): See Sequence No. 2
  n is a or g or c or t
ggtgggaggmtmghndnnnmgdtttaagga

SEQ ID NO: 53 - DNA, 32 bp, Artificial sequence
  Sequence targeted against human Rhinovirus 14. Section I: positions 1 to 8, Section III: positions 24 to 32
  misc_feature (9)..(23): The illustrated core domain represents a preferred core domain embodiment
  misc_feature (9)..(23): See Sequence No. 2
  n is a or g or c or t
gggtgggaggmtmghndnnnmgdtttaaggaa

SEQ ID NO: 54 - DNA, 5 bp, Artificial sequence
  Target sequence on VR1-mRNA
  Target sequence against which a preferred embodiment of the siRNA of the invention is directed
  repeat_unit (3) OTHER INFORMATION: The nucleotice of position 3occurs 19 times in succession
aantt

SEQ ID NO: 55 - DNA, 23 bp, Artificial sequence
  Specific example of a sequence against which the siRNA of the invention is directed
aagcgcaucuucuacuucaactt

SEQ ID NO: 56 - DNA, 23 bp, Artificial sequence
  Specific example of a sequence against which the siRNA of the invention is directed
aaguucgugacaagcauguactt

SEQ ID NO: 57 - DNA, 23 bp, Artificial sequence
  Specific example of a sequence against which the siRNA of the invention is directed
aagcauguacaacgagaucuutt

SEQ ID NO: 58 - DNA, 23 bp, Artificial sequence
  Specific example of a sequence against which the siRNA of the invention is directed
aaccgucaugacaugcuucuctt

SEQ ID NO: 59 - DNA, 23 bp, Artificial sequence
  Specific example of a sequence against which the siRNA of the invention is directed
aagaauaacucucugccuaugtt

SEQ ID NO: 60 - DNA, 23 bp, Artificial sequence
  Specific example of a sequence against which the siRNA of the invention is directed
aauguggguaucaucaacgagtt

SEQ ID NO: 61 - DNA, 21 bp, Artificial sequence
  Sense-strand of a duplex molecule of an especially preferred example of an siRNA-construct of the invention
  Sense-strand of the construct VsiRNA1
gcgcaucuucuacuucaactt

SEQ ID NO: 62 - DNA, 21 bp, Artificial sequence
  Antisense-strand of a duplex molecule of an especially preferred example of an siRNA-construct of the invention
  Antisense-strand of the constuct VsiRNA1
guugaaguagaagaugcgctt

SEQ ID NO: 63 - DNA, 21 bp, Artificial sequence
  Sense-strand of a duplex molecule of an especially preferred example of an siRNA-construct of the invention
  Sense-strand of the construct VsiRNA2
guucgugacaagcauguactt

SEQ ID NO: 64 - DNA, 21 bp, Artificial sequence
  Antisense-strand of a duplex molecule of an especially preferred example of an siRNA-construct of the invention
  Antisense-strand of the construct VsiRNA2
guacaugcuugucacgaactt

SEQ ID NO: 65 - DNA, 21 bp, Artificial sequence
  Sense-strand of a duplex molecule of an especially preferred example of an siRNA-construct of the invention
  Sense-strand of the construct VsiRNA3
gcauguacaacgagaucuutt

SEQ ID NO: 66 - DNA, 21 bp, Artificial sequence
  Antisense-strand of a duplex molecule of an especially preferred example of an siRNA-construct of the invention
  Antisense-strand of the construct VsiRNA3
aagaucucguuguacaugctt

SEQ ID NO: 67 - DNA, 21 bp, Artificial sequence
  Sense-strand of a duplex molecule of an especially preferred example of an siRNA-construct of the invention
  Sense-strand of the construct VsiRNA4
ccgucaugacaugcuucuctt

SEQ ID NO: 68 - DNA, 21 bp, Artificial sequence
  Antisense-strand of a duplex molecule of an especially preferred example of an siRNA-construct of the invention
  Antisense-strand of the construct VsiRNA4
gagaagcaugucaugacggtt

SEQ ID NO: 69 - DNA, 21 bp, Artificial sequence
  Sense-strand of a duplex molecule of an especially preferred example of an siRNA-construct of the invention
  Sense-strand of the construct VsiRNA5
gaauaacucucugccuaugtt

SEQ ID NO: 70 - DNA, 21 bp, Artificial sequence
  Antisense-strand of a duplex molecule of an especially preferred example of an siRNA-construct of the invention
  Antisense-strand of the construct VsiRNA5
cauaggcagagaguuauuctt

SEQ ID NO: 71 - DNA, 21 bp, Artificial sequence
  Sense-strand of a duplex molecule of an especially preferred example of an siRNA-construct of the invention
  Sense-strand of the construct VsiRNA6
uguggguaucaucaacgagtt

SEQ ID NO: 72 - DNA, 21 bp, Artificial sequence
  Antisense-strand of a duplex molecule of an especially preferred example of an siRNA-construct of the invention
  Antisense-strand of the construct VsiRNA6
cucguugaugauacccacatt

SEQ ID NO: 73 - DNA, 31 bp, Artificial sequence
  OTHER INFORMATI ON: D
atgtcgtggggctagctacaacgagattagg

SEQ ID NO: 74 - DNA, 29 bp, Artificial sequence
  DV15-7
gtcgtggggctagctacaacgagattagg

SEQ ID NO: 75 - DNA, 33 bp, Artificial sequence
  DH5-9
ccggggaaaggctagctacaacgaagaagtgct

SEQ ID NO: 76 - DNA, 31 bp, Artificial sequence
  DH5-8
cggggaaaggctagctacaacgaagaagtgc

SEQ ID NO: 77 - DNA, 29 bp, Artificial sequence
  DH5-7
ggggaaaggctagctacaacgaagaagtg

SEQ ID NO: 78 - DNA, 16 bp, Artificial sequence
  Partial sequence of the type 10-23 DNA-enzyme (Fig. 1)
rggctagctacaacga

SEQ ID NO: 79 - DNA, 33 bp, Artificial sequence
  misc_feature (10) ... (23): General description of a type 10-23 DNA-enzyme with the consensus sequence of Section II according to the invention (Fig. 3)
  misc_feature (9) ... (9): R denotes a nucleotide that preferably contains a purine base, Y denotes a nucleotide that preferably contains a pyrimidine base
  misc_feature (9) ... (24): R is a nucleotide that is complementary to Y, the respective nucleotides N of positons 1 to 8 and 25 to 33 are complementary to the respective nucleotides N of the RNA-strand that is to be cleaved
  n is a or g or c or t
nnnnnnnnrggmtmghndnnnmgdnnnnnnnnn

SEQ ID NO: 80 - RNA, 19 nt, Artificial sequence
  misc_feature (1) ... (19): RNA substrate to be cleaved (Fig. 3)
  n is a or g or c or u
nnnnnnnnnrynnnnnnnn

SEQ ID NO: 81 - DNA, 18 bp, Artificial sequence
  V15 (Antisense-oligonucleotide)
catgtcatgacggttagg

SEQ ID NO: 82 - DNA, 18 bp, Artificial sequence
  V30 (Antisense-oligonucleotide)
atcttgttgacggtctca

SEQ ID NO: 83 - DNA, 18 bp, Artificial sequence
  V15inv (Inverted sequence to V15)
ggattggcagtactgtac

SEQ ID NO: 84 - DNA, 18 bp, Artificial sequence
  V30inv (Inverted sequence to V30)
actctggcagttgttcta

(19) United States
(12) Patent Application Publication
(10) Pub. No.: US 2006/0122136 A1
(43) Pub. Date: Jun. 8, 2006

(54) Effective and stable DNA enzymes

(75) Inventors: Steffen Schubert, Berlin (DE); Jens Kurreck, Berlin (DE); Arnold Gruenweller, Berlin (DE); Volker Erdmann, Berlin (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/123,097

(22) Filed: May 6, 2005

Related U.S. Application Data
(63) Continuation of application No. PCT/EP03/12413, filed on Nov. 6, 2003.

(30) Foreign Application Priority Data
Nov. 6, 2002 (DE) 102 51 682.0
May 15, 2003 (DE) 103 22 662.1

(51) Int. Cl. A61K 48/00; C12Q 1/68; C07H 21/02; C12P 21/06; C12N 9/22

(52) U.S. Cl. 514/44; 435/6; 435/69.1; 435/199; 435/320.1; 435/325; 536/23.1

(57) ABSTRACT

The present invention relates to DNA enzymes of type 10-23 with certain modifications of specific nucleotides in the core sequence rendering the DNA enzymes particularly stable and additionally exhibiting substantially the same or a higher cleavage efficiency with respect to their substrate when compared against the corresponding unmodified DNA enzymes. The present application further provides host cells containing the DNA enzymes according to the invention. In addition there is provided a pharmaceutical formulation which contains the DNA enzymes or host cells according to the invention. The DNA enzymes and further subjects are directed in particular against the vanilloid receptor 1 (VR1), or picornaviruses. The present invention further provides small interference RNA molecules (siRNA) directed against VR1, and host cells containing the siRNA. The siRNA and corresponding host cells are suitable as pharmaceutical formulations or for the preparation of pharmaceutical formulations, in particular for the treatment of pain and other pathological conditions associated with VR1.

### CROSS REFERENCE TO RELATED APPLICATIONS

This application is a continuation of International Patent Application No. PCT/EP2003/012413, filed Nov. 6, 2003, designating the United States of America, and published in Germany as WO 2004/042046 A2, the entire disclosure of which is incorporated herein by reference. Priority is claimed based on German Patent Application Nos. 102 51 682.0, filed Nov. 6, 2002, and 103 22 662.1 filed May 15, 2003.

### FIELD OF THE INVENTION

The present invention relates to DNA enzymes and, in particular, modifications of enzymes of type 10-23.

### BACKGROUND OF THE INVENTION

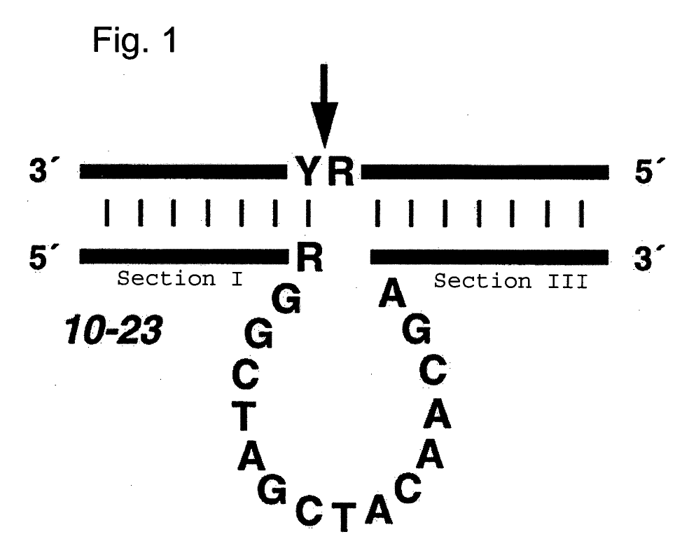

RNA-cleaving DNA enzymes have been developed from hammerhead ribozymes by in vitro selection experiments. Compared with RNA species, DNA enzymes are easier to produce and are more stable, in particular in biological tissues. The DNA enzyme known in the prior art having the greatest cleavage efficiency and the most flexible substrate recognition is the so-called DNA enzyme of type “10-23” (Santoro and Joice (1997) Proc. Natl. Acad. Sci. USA 94: 4262-4266). The DNA enzyme of type 10-23 contains a catalytic domain (core sequence) of 15 nucleotides flanked by two substrate recognition domains or arms each comprising from 7 to 10 nucleotides (see FIG. 1). The DNA enzyme of type 10-23 binds the RNA substrate by base pairing according to the Watson-Crick rules via the substrate recognition arms.

Despite the higher stability of DNA enzymes compared with ribozymes, it is necessary in particular for in vivo applications, for example as pharmaceutical formulations, to stabilize these molecules towards nucleolytic attacks.

### SUMMARY OF THE INVENTION

Accordingly, one object of certain embodiments of the present invention is, on the one hand, to provide DNA enzymes of type 10-23 that exhibit greater stability towards nucleolytic degradation, the catalytic activity towards the particular RNA substrate substantially corresponding to, and preferably being greater than, that of the non-stabilised DNA enzyme.

The present invention relates to DNA enzymes of type 10-23 which, owing to modification of specific nucleotides in the core sequence, are particularly stable and additionally exhibit substantially the same or a higher cleavage efficiency in respect of their substrate as the corresponding unmodified DNA enzymes. The present application further provides host cells containing the DNA enzymes according to the invention. In addition there is provided a pharmaceutical formulation which contains the DNA enzymes or host cells according to the invention. The DNA enzymes and further subjects are directed in particular against the vanilloid receptor 1 (VR1), or picornaviruses. The present invention further provides small interference RNA molecules (siRNA) directed against VR1, and host cells containing the siRNA. The siRNA and corresponding host cells are suitable as pharmaceutical formulations or for the preparation of pharmaceutical formulations, in particular for the treatment of pain and other pathological conditions associated with VR1.

The effective treatment of pain is a great challenge for molecular medicine. Acute and transitory pain is an important signal from the body for protecting humans against severe harm from environmental influences or against excessive strain on the body. Chronic pain, on the other hand, which lasts longer than the cause of the pain and the expected period of healing, has no known biological function. Hundreds of millions of people worldwide are affected by chronic pain. In Germany alone, about 7.5 million people suffer from chronic pain. Pharmacological treatment, in particular of chronic pain, is unsatisfactory at present. The analgesics known in the art are frequently not sufficiently effective and in some cases have serious side-effects.

For this reason new targets, structures occurring naturally in the body, via which a pain-modulating action, for example of low molecular weight active ingredients or other compounds, such as antisense oligodeoxyribonucleotides (ODN), appears possible, are frequently sought for the purposes of treatment, in particular of chronic pain. The vanilloid receptor 1 (VR1) (also known as the capsaicin receptor) cloned by Caterina et al. (1997) Nature 389: 816-824 is a highly promising starting point for the development of new pharmaceutical formulations against pain. This receptor is a cation channel which is expressed predominantly by primary sensory neurones (Caterina et al. (1997), supra). VR1 is activated by capsaicin, a component of chilli pods, heat (>43° C.) and a low pH value (i.e. protons) as a result of tissue damage and causes an influx of calcium into primary afferent neurones. VR1 knockout mice do not develop thermal hyperalgesia following tissue damage or inflammations (Caterina et al. (2000) Science 288: 306-313; Davis et al. (2000) Nature 405: 183-187).

WO 02/18407 discloses antisense-ODN and DNA enzymes of type 10-23 likewise subsumed under that term, which lead to cleavage of VR1 -mRNA.

A further object of the present invention is therefore to provide DNA enzymes of type 10-23 which are directed against the mRNA of the VR1 receptor and exhibit greater stability than the ODN disclosed in the prior art while having comparable or higher catalytic activity.

The picornaviruses include epidemiologically important pathogens, such as rhinoviruses, numerous enteroviruses (including echoviruses, the three polioviruses, various Coxsackie viruses), which cause various diseases, in particular in humans, such as colds (acute rhinitis) or severe chronic inflammations of the nasopharynx (rhinopathies), poliomyelitis, inflammatory cardiac diseases, viral meningitis, etc. It is therefore a further object of the present invention to provide particularly effective and stable DNA enzymes of type 10-23 against picornaviruses.

In recent years, the technique of RNA interference (RNAi) in particular has proved suitable in vitro in some applications as an effective mechanism for switching off genes. RNA interference is based on double-stranded RNA molecules (dsRNA) which trigger the sequence-specific suppression of gene expression (Zamore (2001) Nat. Struct. Biol. 9: 746-750; Sharp (2001) Genes Dev. 5: 485-490; Hannon (2002) Nature 41: 244-251). However, the transfection of mammalian cells with long dsRNA brought about an interferon response by activation of protein kinase R and RNaseL (Stark et al. (1998) Annu. Rev. Biochem. 67: 227-264; He and Katze (2002) Viral Immunol. 15: 95-119). These non-specific effects are avoided if shorter, for example 21- to 23-mer, so-called siRNAs (small interfering RNA), are used, because non-specific effects are not triggered by dsRNA shorter than 30 bp (Elbashir et al. (2001) Nature 411: 494-498). Recently, siRNA molecules have also been used in vivo (McCaffrey et al. (2002) Nature 418: 38-39; Xia et al. (2002) Nature Biotech. 20: 1006-1010; Brummelkamp et al. (2002) Cancer Cell 2: 243-247).

Accordingly, it is a further object of the present invention to provide siRNA molecules which permit the effective treatment of pathogenic conditions associated with VR1, especially of pain.

The objects mentioned above are achieved by the embodiments of the present invention characterised in the claims.

There is provided in particular a DNA enzyme of type 10-23 (referred to as “DNA enzyme” hereinbelow) comprising from 5′ to 3′ a first substrate recognition arm (“section I” hereinbelow), a catalytic core sequence (“section II” hereinbelow) and a second substrate recognition arm (“section III” hereinbelow), wherein one or more of the nucleotides 2, 7, 8, 11, 14 and 15 of section II (which preferably contains 15 nucleotides in total) has/have been modified.

The present invention is based on the surprising finding that chemically modified nucleotides can expediently be introduced into DNA enzymes, in particular those against VR1 or picornaviruses, into the core sequence at positions 2, 7, 8, 11, 14 and/or 15 and, as described further hereinbelow, can also be introduced into the substrate recognition arms (sections I and III), optionally with adaptation of the length of these substrate recognition arms, in order thus to obtain stabilised DNA enzymes that do not exhibit a significant reduction in the catalytic activity or even exhibit an improved catalytic activity towards the particular RNA substrate.

According to the invention the term “modified nucleotide” means that the nucleotide in question has been chemically modified. The term “chemical modification” is understood by a person skilled in the art to mean that the modified nucleotide has been changed compared with naturally occurring nucleotides by the replacement, addition or removal of individual or of a plurality of atoms or atom groups. According to the invention the chemical modification of a nucleotide may therefore concern the ribose (e.g. 2′-O-methyl ribonucleotides, so-called “locked nucleic acid” (LNA) ribonucleotides and inverted thymidine), the phosphoro(di)ester bond (e.g. phosphorothioates, phosphoroamidates, methyl phosphonates and peptide nucleotides) and/or the base (e.g. 7-deazaguanosine, 5-methylcytosine and inosine).

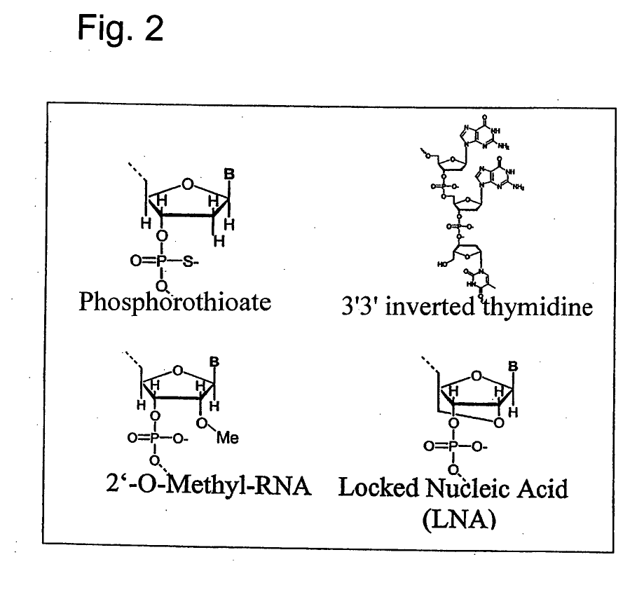

Preferred modified nucleotides according to the present invention are, for example, phosphorothioate nucleotides, inverted thymidine, 2′-O-methyl ribonucleotides and LNA ribonucleotides. These modifications according to the invention are shown by way of example in FIG. 2.

As is shown in FIG. 2, LNAs are ribonucleotides or deoxyribonucleotides that contain a methylene bridge which links the 2′-oxygen of the ribose with the 4′-carbon. An overview of LNAs is given, for example, by Braasch and Corey (2001) in Chem. Biol. 8: 1-7. This article is incorporated by reference in the present disclosure. LNAs are commercially available, for example from Proligo, Boulder, Colo., USA. Phosphorothioates can be obtained, for example, via MWG-Biotech AG, Ebersberg, Germany. 2′-O-Methyl-modified ribonucleotides are obtainable inter alia from IBA-NAPS, Göttingen, Germany.

In preferred DNA enzymes of the present invention all the nucleotides 2, 7, 8, 11, 14 and 15 of section 11 (core sequence) have been modified.

The present invention further provides DNA enzymes in which one or more nucleotides of the substrate recognition domains (or substrate recognition arms), that is to say of section I and/or of section III, has/have been modified, in particular by phosphorothioate, inverted thymidine, 2′-O-methyl ribose or LNA ribonucleotides. Preferred embodiments of the present invention are DNA enzymes in which the modifications of the above definition are present in section II (catalytic core sequence) and in sections I and/or Ill.

In further preferred embodiments of the DNA enzyme according to the invention, all the nucleotides of section I and/or III, in particular all the nucleotides of both sections, have been modified, it being further preferred for all the nucleotides to have been modified by phosphorothioate or 2′-O-methyl ribose.

Furthermore, preferably more than 3, in particular from 3 to 7, more preferably from 3 to 6, in particular from 3 to 5 nucleotides of section I and/or of section III, preferably of both sections, have been modified. Particularly preferred forms of the DNA enzyme according to the invention are obtained when the modified nucleotides are located at the 5′-end of section I and/or at the 3′-end of section III, i.e. at the ends of the DNA enzyme. The modified nucleotides here are preferably 2′-O-methyl ribonucleotides or LNA ribonucleotides.

2′-O-Methyl and LNA ribonucleotides are particularly preferred modifications according to the present invention because these nucleotides effect a higher affinity of the DNA enzyme for the substrate. In particular in the case of substrate excess (i.e. so-called “multiple turnover” conditions), as are to be applied for the effectiveness of the DNA enzyme according to the invention under in vivo conditions, the affinity of the enzyme for the substrate should not be too high because otherwise the product release determines the kinetics. A measure of the affinity of the DNA enzyme according to the invention is its melting point with the substrate. According to the invention it has been found in this respect that an optimum velocity is observed which, for example in the case of DNA enzymes directed against VR1 and rhinovirus 14, is slightly above the reaction temperature (37° C.). According to the invention, therefore, particularly preferred forms of the DNA enzyme are obtained when, in the case of a given target molecule, the melting temperature is appropriately adjusted by varying the modified nucleotides, the nature of the modification and/or the length of the substrate recognition arms (sections I and III). The melting temperature of the double strands formed between sections I and III of the DNA enzyme according to the invention and the corresponding target sequences is from about 33 to about 45° C., more preferably from about 35 to about 42° C., in particular from about 37 to about 40° C., especially about 39° C.

It has been found in particular that preferred embodiments of the DNA enzyme according to the invention are obtained when section I and/or section III, in particular both sections, comprise not more than 9, more preferably not more than 8, in particular 7 nucleotides.

Very particularly preferred DNA enzymes of the present invention are species having the following substrate recognition arm lengths and the following modifications, the first figure indicating the length of the substrate recognition arms and the second figure indicating the number of modified nucleotides, which are preferably located at the end of the arms, and OMe denoting 2′-O-methyl ribonucleotides and LH denoting LNA ribonucleotides:

OMe9-4, OMe8-4, OMe7-3, OMe7-4, OMe7-5, OMe7-6, OMe7-7, OMe7-7, OMe6-5, LH9-4, LH7-3 and LH7-4.

The base sequence of the catalytically active core domain of the DNA enzyme, which was developed by Santoro and Joyce (1997), supra, is from 5′ to 3′ GGCTAGCTACAACGA (see FIG. 1). According to the invention it has further been found that the bases of the core sequence are flexible, in particular at positions 7 to 12, it even being possible to omit thymidine at position 8. It follows from these findings that the DNA enzyme according to the invention may exhibit the following consensus sequence in section 11 from 5′ to 3′:
GGMTMGH(N)DNNNMGD.

Therein M=A or C, H=A, C or T, D=G, A or T and N=any (naturally occurring) base. Bases and nucleotides in brackets do not have to be present.

With regard to the substrate, the DNA enzyme according to the invention is not limited. The DNA enzyme can therefore be used in principle against all mRNA molecules, other RNA, such as viral RNA, viroids, etc. Target sequences are those which exhibit the cleavage motif of the 10-23 DNA enzymes, namely a purine/pyrimidine motif. Preferred target sequences include a GU motif, because GU sequences are cleaved particularly effectively by DNA enzymes.

The DNA enzyme according to the invention is preferably directed against the mRNA of the vanilloid receptor 1 (VR1), especially of mammals, such as humans, apes, dogs, cats, rats, mice, rabbits, guinea pigs, hamsters, cattle, pigs, sheep and goats.

Specific base sequences of sections I and III (from 5′ to 3′) are the following, a sequence differing therefrom in one nucleotide optionally also being possible, with the proviso that the nucleotide differing from the given sequences is not located at one of the last three positions of section I nor at one of the first three positions of section III (N=any base or any nucleotide)

| | |
|---|---|
| Section I | Section III |

| | |
|---|---|
| GTCATGA | GGTTAGG |
| TGTCATGA | GGTTAGGG |
| ATGTCATGA | GGTTAGGGG |
| GTCGTGG | GATTAGG |
| TGTCGTGG | GATTAGG |
| ATGTCGTGG | GATTAGG |
| TTGTTGA | GGTCTCA |
| CTTGTTGA | GGTCTCAC |
| TCTTGTTGA | GGTCTCACC |
| TTGTTGA | AGTCTCA |
| CTTGTTGA | AGTCTCAN |
| TCTTGTTGA | AGTCTCANN |
| GGCCTGA | CTCAGGG |
| CGGCCTGA | CTCAGGGA |
| TCGGCCTGA | CTCAGGGAG |
| TGCTTGA | CGCAGGG |
| CTGCTTGA | CGCAGGGN |
| TCTGCTTGA | CGCAGGGNN |
| GTGTGGA | TCCATAG |
| GGTGTGGA | TCCATAGG |
| TGGTGTGGA | TCCATAGGC |
| ACGTGGA | TGAGACG |
| GACGTGGA | TCAGACGN |
| CGACGTGGA | TCAGACGNN |
| GTGGGGA | TCAGACT |
| GGTGGGGA | TCAGACTC |
| GGGTGGGGA | TCAGACTCC |
| GTGGGTC | GCAGCAG |
| AGTGGGTC | GCAGCAG |
| GAGTGGGTC | GCAGCAG |
| CGCTTGA | AAATCTG |
| GCGCTTGA | AAATCTGT |
| TGCGCTTGA | AAATCTGTC |
| CGCTTGA | GAATCTG |
| GCGCTTGA | GAATCTGN |
| TGCGCTTGA | GAATCTGNN |
| CTCCAGA | ATGTGGA |
| GCTCCAGA | ATGTGGAA |
| AGCTCCAGA | ATGTGGAAT |
| CTCCAGG | AGGTGGA |
| GCTCCAGG | AGGTGGA |
| AGCTCCAGG | AGGTGGA |
| GGTACGA | TCCTGGT |
| GGGTACGA | TCCTGGTA |
| CGGGTACGA | TCCTGGTAG |
| GGTGCGG | TCTTGGC |
| GGGTGCGG | TCTTGGC |
| CGGGTGCGG | TCTTGGC |

Further preferred DNA enzymes of the present invention are directed against virus RNA, in particular against a picornavirus, as disclosed, for example, in Kayser et al., Medizinische Mikrobiologie, 8th Edition, Thieme Verlag, Stuttgart, 1993 (e.g. hepatitis A virus, human enteroviruses, such as polioviruses, Coxsackie viruses and echoviruses, animal enteroviruses, such as the Tschen virus pathogenic for pigs, human rhinoviruses, such as rhinovirus 14 etc. (a person skilled in the art knows more than 80 types), animal rhinoviruses, such as the foot-and-mouth (FAM) virus, and calciviruses, such as the vesicular exanthem virus in pigs).

The preferred target RNA is derived, for example, from (preferably human) rhinovirus 14, particularly advantageous embodiments of the DNA enzyme of the present invention being at least partly complementary in sections I and III to the 5′-untranslated region (5′-UTR). This region comprises a consensus sequence which is conserved in numerous picornaviruses having a group I IRES. Specific examples of sequences according to the invention directed against human rhinovirus 14 and other picornaviruses, in particular those having a group I IRES, are indicated hereinbelow from 5′ to 3′, a sequence differing therefrom in a nucleotide also optionally being possible, with the proviso that the nucleotide differing from the given sequences is not located at one of the last three positions of section I or at one of the first three positions of section III: (N=any base or any nucleotide)

| | |
|---|---|
| Section I | Section III |

| | |
|---|---|
| GTGGGA | TTTAAGG |
| GGTGGGA | TTTAAGGA |
| GGGTGGGA | TTTAAGGAA |

A further embodiment of the present invention is constituted by a siRNA directed against VR1. According to the invention, the term “siRNA” is a double-stranded RNA molecule (dsRNA) that comprises from 19 to 29 bp, in particular from 21 to 23 bp, and has a sequence complementary to the mRNA of VR1. The mRNA of VR1 is preferably derived from mammals, such as humans, apes, rats, dogs, cats, mice, rabbits, guinea pigs, hamsters, cattle, pigs, sheep and goats. Preferred embodiments of the siRNA according to the invention are directed against target sequences of VR1-mRNA that begin with AA, have a GC content of less than 50% and/or are unique in the genome and accordingly occur only in the target gene.

A particularly preferred siRNA of the present invention is directed against a target sequence of VR1 -mRNA exhibiting the general structure 5′-AA(N₁₉)TT-3′ (wherein N stands for any desired base). In principle, the siRNA may be directed against any section of the VR1 -mRNA, in particular against coding sections, but optionally also against non-coding sections (5′- or 3′-terminal of the coding region) or in the overlapping region between the coding and the non-coding region. However, a siRNA according to the invention may also be directed against target sequences in the primary transcript of VR1.

In particular, the siRNA according to the invention is directed against sequences selected from the group consisting of 5′-AAGCGCAUCUUCUACUUCAACTT-3′, 5′-AAGUUCGUGACAAGCAUGUACTT-3′, 5′-AAGCAUGUACAACGAGAUCUUTT-3′, 5′-AACCGUCAUGACAUGCUUCUCTT-3′, 5′-AAGMUAACUCUCUGCCUAUGTT-3′ and 5′-MUGUGGGUAUCAUCAACGAGTT-3′.

Particularly preferred siRNA species of the present invention are the following duplex molecules:

### Sense Strand/Antisense Strand

5′-GCGCAUCUUCUACUUCAACdTdT-3′/5′-GUUGAAGUAGAAGAUGCGCdTdT-3′
5′-GUUCGUGACAAGCAUGUACdTdT-3′/5′-GUACAUGCUUGUCACGAACdTdT-3′
5′-GCAUGUACAACGAGAUCUUdTdT-3′/5′-AAGAUCUCGUUGUACAUGCdTdT-3′
5′-CCGUCAUGACAUGCUUCUCdTdT-3′/5′-GAGAAGCAUGUCAUGACGGdTdT-3′
5′-GAAUAACUCUCUGCCUAUGdTdT-3′/5′-CAUAGGCAGAGAGUUAUUCdTdT-3′
5′-UGUGGGUAUCAUCAACGAGdTdT-3′/5′-CUCGUUGAUGAUACCCACAdTdT-3′

Very particular preference is given to the above siRNA whose sense strand exhibits the sequence 5′-GCGCAUCUUCUACUUCAACdTdT-3′ and whose antisense strand exhibits the sequence 5′-GUUGAAGUAGAAGAUGCGCdTdT-3′.

siRNA molecules can be obtained from various suppliers, for example IBA GmbH (Göttingen, Germany).

According to preferred embodiments, the siRNA of the present invention is in chemically modified form, in particular in order to avoid premature degradation by nucleases. The comments made above in connection with the DNA enzymes according to the invention apply correspondingly in this respect, in particular, for example, the use of phosphorothioates (as disclosed in Eckstein (Antisense Nucleic Acid Drug Dev., 10 117, 2000) and incorporated by reference in the present disclosure). In the siRNA according to the invention it is further possible for the hydroxyl group at the 2′-position to be modified in order to achieve higher stability. In this respect, the disclosure made in Levin, Biochim. Biophys Acta, 1489, 69, 1999) is incorporated by reference in the present disclosure.

According to the invention it has been found, surprisingly, that the subjects according to the invention, especially the above-defined siRNA, suppress the expression of VR1 to a greater extent than do conventional antisense oligonucleotides. The siRNA according to the invention especially proves to be particularly effective especially in vivo and is superior, for example, to (conventional) unmodified and modified (phosphorothioate, 2′-O-methyl RNA, LNA (LNA/DNA gapmers)) antisense DNA oligonucleotides.

The present invention further provides host cells, with the exception of human germ cells and human embryonal stem cells, which have been transformed with at least one DNA enzyme according to the invention and/or at least one siRNA according to the invention. DNA enzymes and siRNA molecules according to the invention can be introduced into the host cell in question by conventional methods, for example transformation, transfection, transduction, electroporation or particle gun. There come into consideration as host cells any cells of prokaryotic or eukaryotic nature, for example cells from bacteria, fungi, yeasts, plant or animal cells. Preferred host cells are bacterial cells, such as Escherichia coli, Streptomyces, Bacillus or Pseudomonas, eukaryotic microorganisms, such as Aspergillus or Saccharomyces cerevisiae, or the conventional baker's yeasts (Stinchcomb et al. (1997) Nature 282: 39).

In a preferred embodiment, however, the cells chosen for transformation by DNA enzymes or siRNA constructs according to the invention are cells from multicellular organisms. In principle, any higher eukaryotic cell culture is available as host cell, although cells of mammals, for example apes, rats, hamsters, mice or humans, are very particularly preferred. A large number of established cell lines is known to the person skilled in the art. The following cell lines are mentioned without implying any limitation: 293T (embryo kidney cell line) (Graham et al., J. Gen. Virol. 36: 59 (1997)), BHK (baby hamster kidney cells), CHO (cells from hamster ovaries, Urlaub and Chasin, Proc. Natl. Acad. Sci. USA 77: 4216, (1980)), HeLa (human carcinoma cells) and further cell lines—in particular cell lines established for laboratory use—for example HEK293, SF9 or COS cells. Very particular preference is given to human cells, especially neuronal stem cells and cells of the “pain pathway”, preferably primary sensory neurones. Following transformation (especially ex vivo transformation) with DNA enzymes or siRNA molecules according to the invention, human cells, especially autologous cells of a patient, are very particularly suitable as pharmaceutical formulations, for example for the purposes of gene therapy, that is to say after cell removal, optional ex vivo expansion, transformation, selection and subsequent re-transplantation into the patient.

The combination of a host cell and a DNA enzyme according to the invention and/or a siRNA according to the invention suitable for the host cell forms a system that can be used to apply the DNA enzymes or siRNA molecules according to the invention.

Accordingly, the subjects according to the invention are suitable as pharmaceutical formulations, for example for inhibiting nociception, for example by reducing expression of the VR1 receptor by means of DNA enzymes and/or siRNA according to the invention.

Consequently, the present invention also includes the use of the subjects mentioned above in the treatment of or in the preparation of a pharmaceutical formulation for the treatment and/or prevention of pain, in particular acute or chronic pain, and also their use in the treatment of or in the preparation of a pharmaceutical formulation for the treatment of sensitivity disorders associated with the VR1 receptor, for example in the treatment of hyperalgesia, neuralgia and myalgia, and of all diseases and symptoms of diseases associated with VR1, especially urinary incontinence, neurogenic bladder symptoms, pruritus, tumours and inflammations.

Pharmaceutical formulations according to the invention, or pharmaceutical formulations prepared using the subjects according to the invention, optionally comprise, in addition to the subjects defined above, one or more suitable auxiliary substances and/or additives. Pharmaceutical formulations according to the invention may be administered as a liquid pharmaceutical formulation form in the form of an injection solution, drops or juices, or as semi-solid pharmaceutical formulation forms in the form of granules, tablets, pellets, patches, capsules, plasters or aerosols and contain, in addition to the at least one subject according to the invention, depending on the galenical form, optionally carriers, fillers, solvents, diluents, colourings and/or binders. The choice of auxiliary substances, and the amounts thereof to be used, depend on whether the pharmaceutical formulation is to be administered orally, perorally, parenterally, intravenously, intraperitoneally, intradermally, intramuscularly, intranasally, buccally, rectally or topicallly, to the mucosa, the eyes, etc. Preparations in the form of tablets, dragées, capsules, granules, drops, juices and syrups are suitable for oral administration; solutions, suspensions, readily reconstitutable dry preparations and sprays are suitable for parenteral and topical administration and for administration by inhalation. Subjects according to the invention in a depot in dissolved form or in a plaster, optionally with the addition of agents promoting penetration of the skin, are suitable percutaneous forms of administration. Forms of preparation for administration orally or percutaneously may release the subjects according to the invention in a delayed/retarded manner. The amount of active ingredient to be administered to a patient varies in dependence on the weight of the patient, the mode of administration, the indication and the severity of the disease. Usually, from 2 to 500 mg/kg body weight of at least one subject according to the invention are administered. If the pharmaceutical formulation is to be used in particular for gene therapy, recommended suitable auxiliary agents or additives are, for example, a physiological saline solution, stabilisers, protease or DNAse inhibitors, etc.

Suitable additives and/or auxiliary substances are, for example, lipids, cationic lipids, polymers, liposomes, nucleic acid aptamers, peptides and proteins (e.g. tet, transportin, transferrin, albumin or ferritin), preferably those that are bonded to DNA or RNA (or synthetic peptide/DNA molecules), in order, for example, to increase the incorporation of nucleic acids into the cell, to direct the pharmaceutical formulation mixture to only one sub-group of cells, to prevent degradation of the nucleic acid according to the invention in the cell, to facilitate storage of the pharmaceutical formulation mixture prior to use, etc. Examples of peptides and proteins or synthetic peptide/DNA molecules are antibodies, antibody fragments, ligands, adhesion molecules, all of which may be modified or unmodified. Examples of auxiliary substances which, for example, stabilise the DNA enzymes and/or siRNA in the cell are nucleic acid-condensing substances such as cationic polymers, poly-L-lysine or polyethyleneimine.

In the case of local administration of subjects according to the invention, for example of DNA enzymes or siRNA constructs according to the invention, administration is effected by injection, catheter, suppository, aerosols (nasal or oral spray, inhalation), trocars, projectiles, pluronic gels, polymers that continuously release pharmaceutical formulations, or any other device permitting local admission. Ex vivo application of the pharmaceutical formulation mixture according to the invention, which is used in the treatment of the above-mentioned indications, also permits local admission.

Subjects according to the invention may optionally be combined in a composition in the form of a pharmaceutical formulation (active ingredient) mixture with, for example, at least one further pain-relieving agent or antiviral agent and/or other agent for the treatment of diseases associated with (rhino)viral infections.

In this manner, subjects according to the invention may be combined, for example, with opiates and/or synthetic opioids (e.g. morphine, levomethadone, codeine, tramadol, bupremorphine) and/or NSAIDs (e.g. diclofenac, ibuprofen, paracetamol), for example in one of the forms of administration disclosed above or, in the course of combined therapy, in separate forms of administration, optionally with different finished forms (formulations), in a medically meaningful therapy plan adapted to the needs of the patient in question. Preference is given to the use of such compositions in the form of pharmaceutical formulation mixtures with, for example, established analgesics for the treatment (or for the preparation of pharmaceutical formulations for the treatment) of the medical indications disclosed herein.

The DNA enzymes and siRNA molecules according to the invention can be prepared by a process known to the person skilled in the art. The corresponding nucleotides are synthesised, for example, in the manner of a Merrifield synthesis on an insoluble carrier (H. G. Gassen et al. (1982) Chemical and Enzymatic Synthesis of Gene Fragments, Verlag Chemie, Weinheim) or in another manner (Beyer and Walter (1984) Lehrbuch der organischen Chemie, page 816 ff., 20th Edition, S. Hirzel Verlag, Stuttgart). The subjects according to the invention may likewise be synthesised by known processes in situ on glass, plastics or metal, for example gold.

The present invention further provides a process for inhibiting the expression of a gene, which process comprises introducing one of the subjects according to the invention, in particular the above-defined DNA enzyme and/or siRNA, into a cell that expresses the gene in question. A preferred target gene of the process according to the invention is the VR1 gene, a DNA enzyme directed against the mRNA of VR1 and/or a corresponding siRNA accordingly being introduced into the cell.

The introduction of the subjects according to the invention into the cell can be carried out in the manner described above.

The Figures Show

FIG. 1 is the schematic representation of a DNA enzyme of type 10-23 according to Santoro et al. (1997), supra, (FIG. 2, p. 4264) (including the RNA substrate). The upper strand, indicated by an arrow, is the RNA strand to be cleaved, the arrow indicating the cleavage site. The lower strand is a representation of the DNA enzyme. In relation to preferred embodiments of the DNA enzyme according to the invention, Y is=U in the upper strand and R=G.

The cleavage site in the upper strand is therefore a so-called GU cleavage site, which is cleaved particularly effectively by DNA enzymes. Correspondingly, R is=A in the lower strand. This is followed 5′-wards, for example, by the further nucleotides from the above-defined section I. In section III, the unpaired A adjacent to section III is followed directly from the 5′ direction 3′-wards by the nucleotides of the second part of the DNA enzyme, complementary to the RNA substrate, for example the sequences mentioned above. Section I and section III accordingly bind the substrate and are therefore referred to as substrate recognition arms of the DNA enzyme.

FIG. 2 shows the structures of phosphorothioate nucleotides, inverted thymidine, 2′-O-methyl ribonucleotides and LNA ribonucleotides.

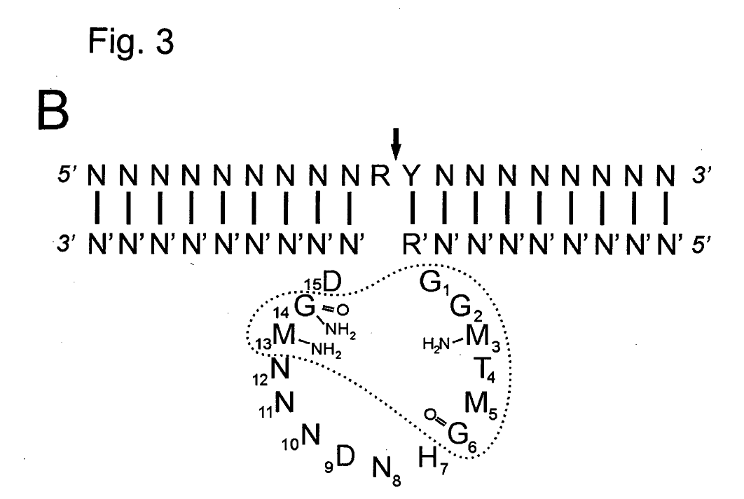

FIG. 3 shows, in a representation corresponding to FIG. 1, the general structure of a DNA enzyme of type 10-23 with the consensus sequence according to the invention of section II (catalytic core sequence). Non-essential nucleotides, which can be replaced by any other nucleotide without any substantial loss of activity, are designated N, N′ stands for any nucleotide complementary to N. R denotes a nucleotide that preferably contains a purine base. Y stands for a nucleotide that preferably contains a pyrimidine base. R′ stands for a nucleotide complementary to Y that contains a purine. M stands for A or C, H stands for A, C or T, and D stands for G, A or T. The region that is probably directly involved in the formation of the catalytic centre is marked with a dotted line. Exocyclic functional groups, which are necessary for the activity of the DNA enzyme, are indicated in each case.

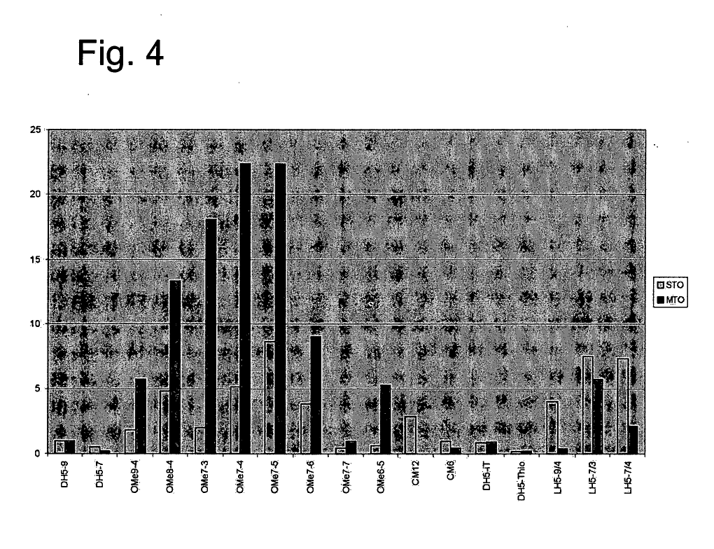

FIG. 4 shows, in a graphic representation, the relative activities of modified DNA enzymes compared with the 5′-UTR of human rhinovirus 14 (DH 5), in each case with a 10-fold excess of DNA enzymes (“single turnover” conditions, STO, left-hand bar in each case) and a 10-fold substrate excess (“multiple turnover” conditions, MTO, right-hand bar in each case). The activities are normalised to the unmodified DNA enzyme with substrate recognition arms having a length of 9 nucleotides. The indicated DNA enzymes exhibit the following modifications: phosphorothioate (Thio), inverted thymidine (iT), 2′-O-methyl RNA (OMe) and LNA (LH). The first figure means the length of the substrate recognition arms, while the second figure indicates the number of modified nucleotides at the end of the arms. CM6 is a DNA enzyme according to the invention in which nucleotides 2, 7, 8, 11, 14 and 15 of the core sequence have been modified (2′-O-methyl ribonucleotides). CM12 is a comparison construct in which 12 nucleotides of the core sequence (all apart from positions 3, 5 and 10) have been modified. DH5-9 denotes the unmodified DNA enzyme against the 5′-UTR of human rhinovirus 14 with substrate recognition arms having a length of 9 nucleotides, while DH5-7 characterises the corresponding DNA enzyme with substrate recognition arms having a length of 7 nucleotides. With an optimum design of the substrate recognition arms (see OMe7-4 and OMe7-5) it is possible to increase the activity of the DNA enzyme under MTO conditions, which are critical, for example, for in vivo use, by a factor of more than 20. The enzyme in which the mentioned 6 nucleotides in the core sequence have together been replaced by the corresponding 2′-O-methyl ribonucleotides is still substantially as active as the unmodified DNA enzyme (see CM6 and DH 5-9). The DNA enzyme in which 12 nucleotides of the core sequence have been modified cleaves the complete 5′-UTR with good effectiveness under STO conditions but is inactive in respect of the complete (long) target under MTO conditions (see CM12). If the nucleotides replaced together in CM12 are each modified individually, the corresponding constructs cleave a 19-mer short section from the target RNA (5′-UTR of human rhinovirus 14) which is complementary to the substrate recognition arms (not shown). The bars in each case show the mean of three independent experiments.

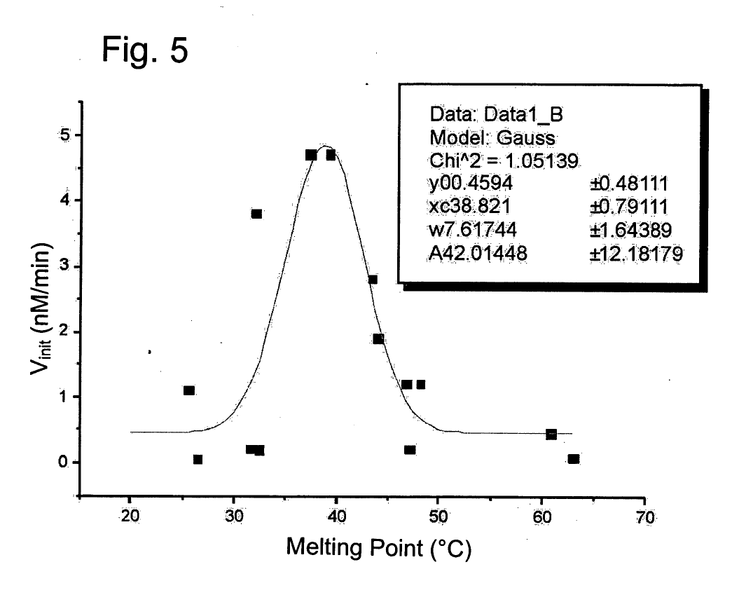

FIG. 5 shows in a diagram the dependence of the initial velocity of substrate cleavage (vᵢₙᵢₜ) on the melting point of the helices formed in a DNA enzyme between sections I and III (substrate recognition arms) with the target sequence in the case of the DNA enzymes studied according to FIG. 4. According to this, an optimum of the initial velocity of the melting point, which is slightly above the reaction temperature (37° C.), is to be determined. In the present case, an optimum melting temperature of 39° C. is found.

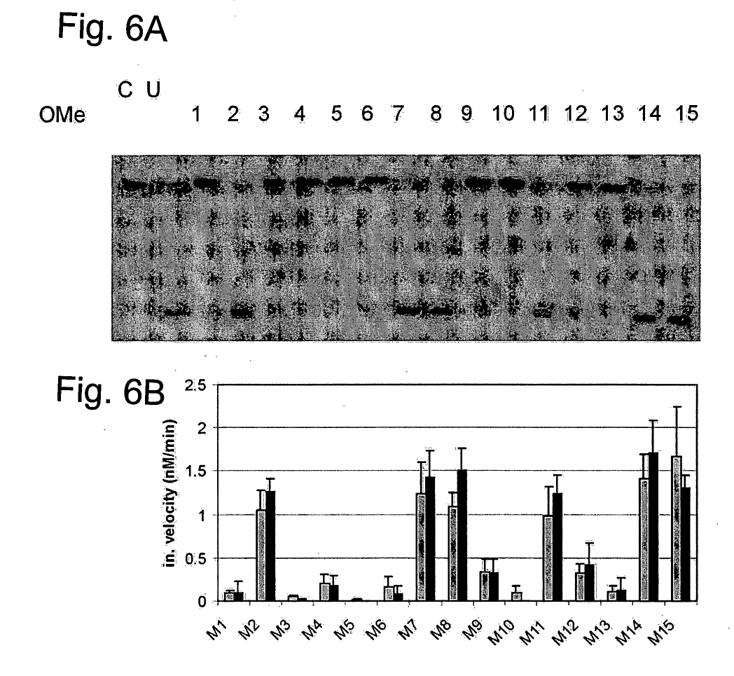

FIG. 6 shows in FIG. 6A the results of an experiment based on a one-hundred-fold excess of the short target RNA having a length of 19 nucleotides. For this purpose, this excess was incubated with DNAzymes at 37° C. for 20 minutes. The uncleaved substrate (upper band) and the 5′-cleavage product (lower band) are to be seen. The following are plotted in the individual traces: trace 1: control without enzyme, trace 2: unmodified DH5, traces 3 to 17 (denoted 1 to 15 in the diagram): DNAzymes with 2′-O-methyl RNA at one of positions 1 to 15 in each case (in corresponding numerical order). FIG. 6B shows in a graphic representation the relative cleavage activities of DNA enzymes against the 5′-UTR of human rhinovirus 14, in which one nucleotide of the core sequence (positions 1 to 15, corresponding to samples M1 to M15) has in each case been modified individually by 2′-O-methyl modifications. The respective activity is normalised to the corresponding unmodified DNA enzyme with recognition arms having a length of 9 nucleotides. Accordingly, nucleotides 2, 7, 8, 11, 14 and 15 can be modified without any loss of activity (with a gain in activity). The bars each show the mean of three independent experiments with the indicated standard deviation, for the short target molecules (grey bar) and the long target molecules (black bar). The height of the bar indicates the percentage cleavage of target RNA by DNAzymes (DNA enzymes).

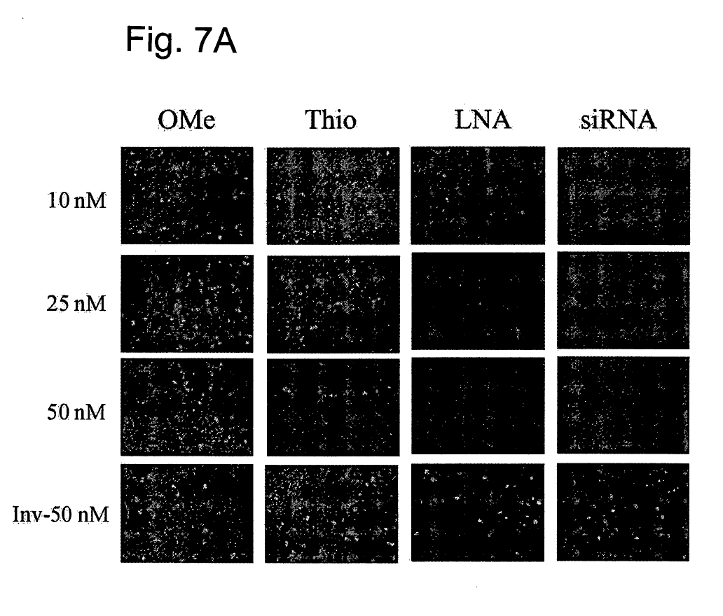

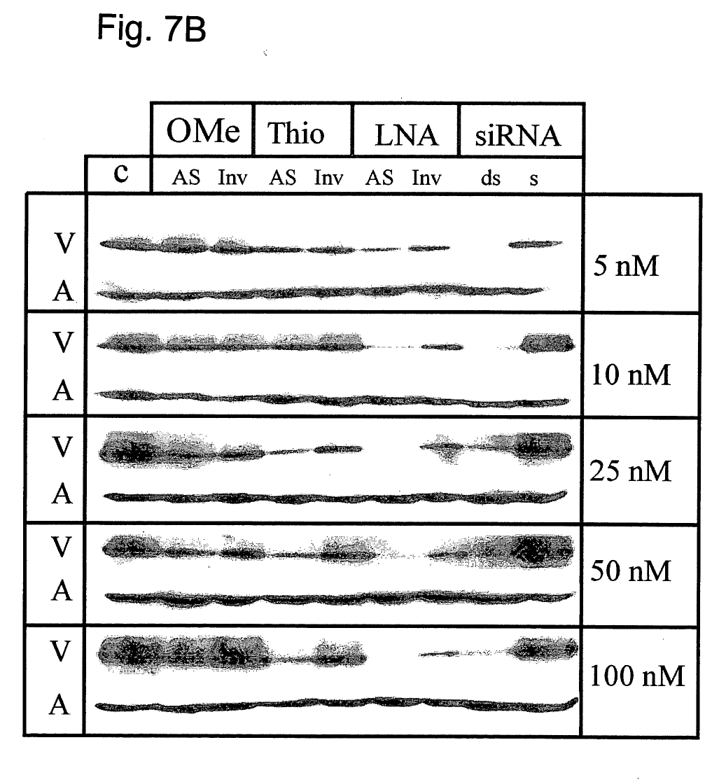

FIG. 7 shows fluorescence microscopic images (A) and corresponding Western blot analyses (B) of cells which, in order to compare the activities of constructs in respect of the inhibition of the expression of VR1, have been cotransformed with a plasmid coding for VR1-GFP and different antisense and siRNA constructs. 2′-O-Methyl RNA, LNA gapmer, phosphorothioate RNA or siRNA was used in concentrations of from 10 to 50 nM. Inverted oligonucleotides for each modification and the sense strand of the siRNA were used as controls and were used in a concentration of 50 nM. As the fluorescence microscopic analysis in (A) shows, the siRNA according to the invention suppresses VR1-GFP expression completely at a concentration of only 10 nM, while LNA gapmer leads to substantially complete suppression of the expression of VR1-GFP only at a concentration of 25 nM. Partial inhibition of VR1-GFP expression is observed in the case of the phosphorothioate antisense ODN at a higher concentration, while the 2′-O-methyl-modified ODN did not bring about any reduction in the expression of the VR1-GFP construct in the observed concentration range. The Western blot analysis (B) confirms the fluorescence microscopic experiments (V: band of the VR1-GFP construct; A: band of actin (control)).

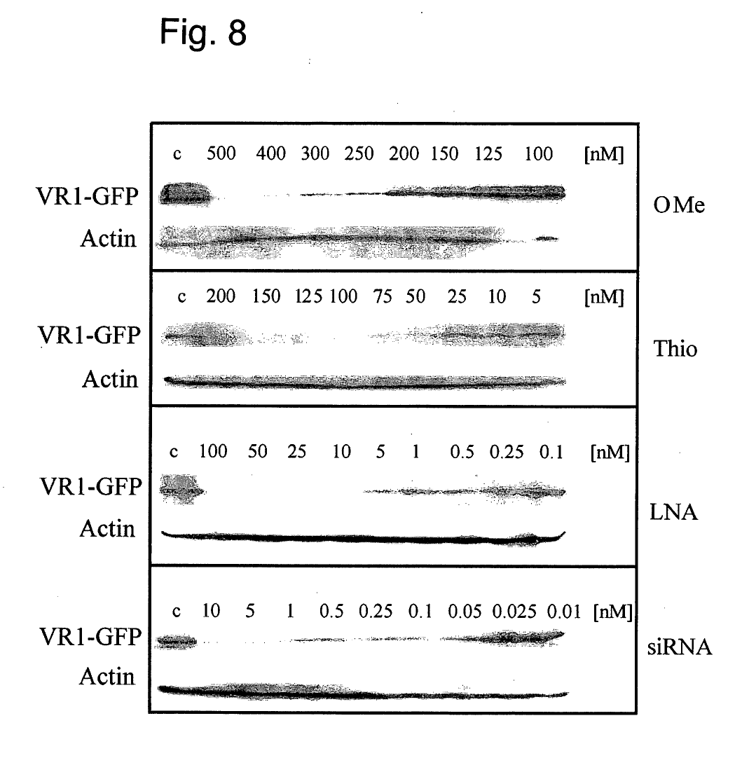

FIG. 8 shows a Western blot analysis of cells which have been cotransformed as described in FIG. 7 but in this case at optimal concentrations for each construct, in order to compare more thoroughly the efficiency of the inhibition of VR1 -GFP expression of siRNA according to the invention with antisense ODN. The actin band served as control.

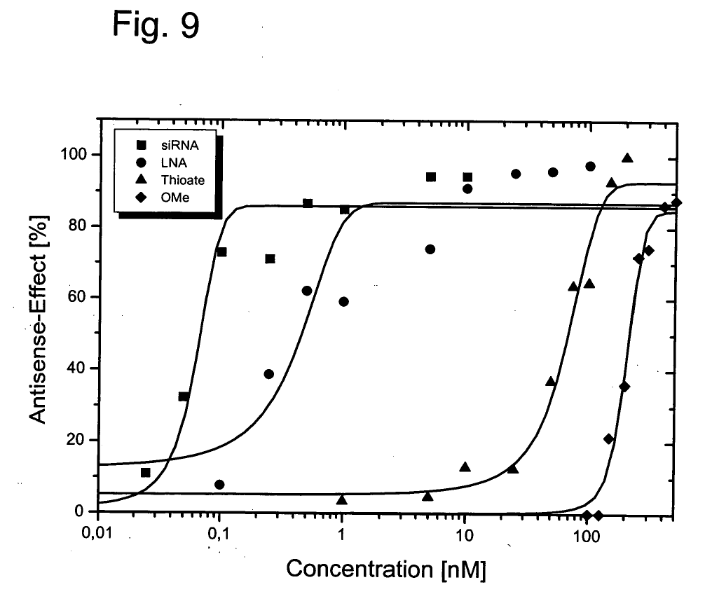

FIG. 9 shows a graphic representation of the quantitative evaluation of the Western blot analyses corresponding to FIG. 8. The protein contents in the individual traces were determined using the Quantity One program. The means of three independent experiments, adapted to a sigmoid curve, are shown in each case. By means of this quantitative evaluation it was possible to assign to the respective inhibitors estimated values of the concentration required for 50% suppression of VR1-GFP expression (IC₅₀ value) (see also Table 3 below).

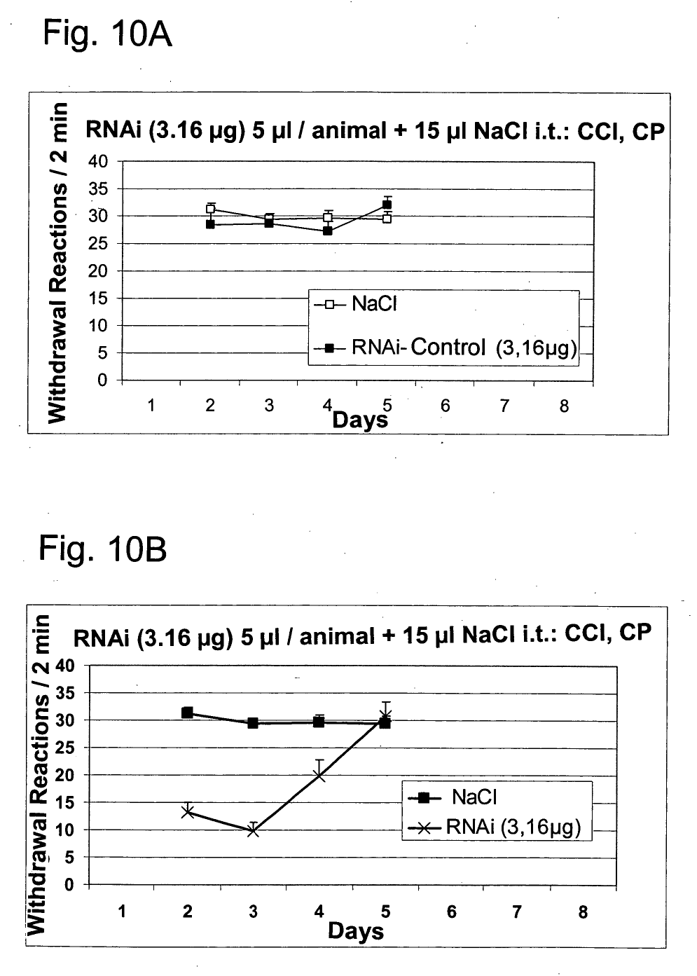

FIG. 10 shows graphic representations of the results of experiments relating to the in vivo analgesic effectiveness of siRNA according to the invention (A) compared with control RNA (B), animals treated with pure NaCl solution being used as control in each case. The rat pain model according to Bennett was used. The pull-away reactions of the injured paw counted within a period of 2 minutes are plotted in each case in dependence on the time after the operation (day of the operation=day 1). The mean values and standard deviations of groups comprising 9 or 10 animals are shown in each case. The administration of siRNA according to the invention brought about a reduction in the pull-away reactions on days 2, 3 and 4 from about 30 to 13, 10 or 20 (A), while the control RNA (sense strand of siRNA) exhibited no significant analgesic action (B).

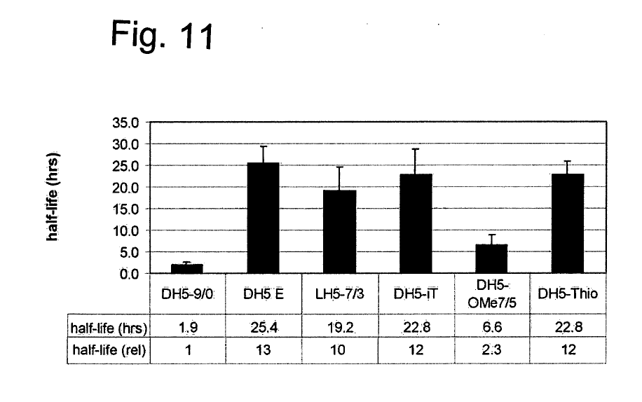

FIG. 11 FIG. 11 shows the stability of modified DNA enzymes in the cell culture medium. To that end, the DNA enzymes were incubated at a final concentration of 1 μM in DMEM with 10% foetal calf serum. Aliquots were removed at specific times, and the remaining amount of full-length oligonucleotides was determined by polyacrylamide gel electrophoresis. Average half-lives and half-lives, normalised relative to unmodified DNAzyme, from three experiments are indicated. The meanings of the abbreviations are given in Table 4. Modifications of the binding arms with LNA nucleotides and phosphorothioates increased the half-life from 2 hours to more than about 20 hours. An inverted thymidine at the 3′-end increased the stability by a factor of 10. 2′-O-Methyl modifications provided solely at the binding arms proved to be markedly inferior to other modifications (half-lives of about 6.5 hours). The newly designed DNAzyme DH5 E (with 2′-O-methyl nucleotides on both binding arms and the catalytic centre) proved to be extremely resistant to degradation. The half-life was increased to 25 hours.

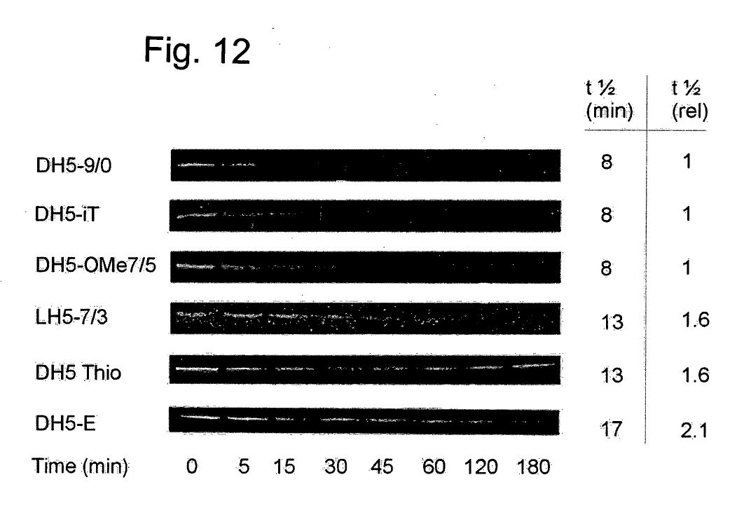

FIG. 12 FIG. 12 shows a comparison of the stability of DNAzymes towards S1 endonuclease. DNAzymes were incubated at a concentration of 2 μM with 0.4 U of S1 endonuclease per 100 pmol of DNAzyme. Aliquots were removed at appropriate times. Full-length and degraded oligonucleotides were separated on a 20% denaturing polyacrylamide gel. Half-lives and relative stability, normalised relative to unmodified DNAzyme, are indicated. As can be seen from FIG. 12, unmodified DNAzyme and DNAzymes with inverted thymidine and 2′-O-methyl RNA “end-blocks” are degraded almost completely after 30 minutes with a half-life of about 8 minutes. The oligonucleotides containing LNA monomers are stained only weakly by ethidium bromide. It can be seen that the DNAzyme stabilised by the introduction of LNAs into the substrate recognition arms was more stable towards S1 endonuclease than the unmodified DNAzyme. DH5-Thio exhibited a two-phase degradation curve, which is due to the chiral nature of the modified nucleotides. About 50% of the starting amount is degraded by the endonuclease with a half-life of about 13 minutes. The other half is subject to a much slower degradation constant. The optimised DNAzyme with 2′-O-methyl monomers in the catalytic core and in the substrate recognition arms has the longest half-life of all the modified enzymes tested. Its stability is increased more than two-fold compared with the unmodified DNAzyme.

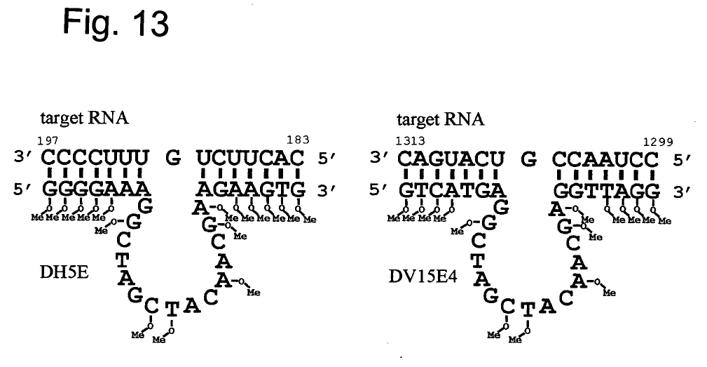

FIG. 13 FIG. 13 shows the modifications according to the invention of optimised DNAzymes. The left-hand representation in FIG. 13 shows DNAzymes against the 5′-NTR (non-translated region) of HRV14 (DH5) and against VR1 mRNA (DV15) on the right-hand side and their respective substrate RNAs. The position of the target RNAs which are bonded by the DNAzymes is indicated, and the nucleotides in which 2′-O-methyl RNA monomers were introduced into the DNAzymes are labelled.

The following Examples illustrate the present invention in greater detail without limiting it.

### EXAMPLE 1

DNA Enzymes

DNA enzymes having the following sequences were studied:

1. DNA enzymes against VR1

DV15-9: ATGTCGTGGGGCTAGCTACAACGAGATTAGG
DV15-1: GTCGTGGGGCTAGCTACAACGAGATTAGG
(substrate recognition arms underlined)

DV1 5 constructs are directed against the 15th GUC of the mRNA of human VR1.

2. DNA enzymes against human rhinovirus 14

DH5-9: CCGGGGAAAGGCTAGCTACAACGAAGAAGTGCT
DH5-8: CGGGGAAAGGCTAGCTACAACGAAGAAGTGC
DH5-7: GGGGAAAGGCTAGCTACAACGAAGAAGTG
(substrate recognition arms underlined)

DH5 constructs are directed against the 5′-UTR of human rhinovirus 14, a consensus sequence which is homologous between numerous picornaviruses having a group I IRES.

Substrates

1. VR1 mRNA

VR1 mRNA was prepared by in vitro transcription. The cDNA of VR1 was first cloned into the vector pcDNA3.1 (+) (Invitrogen). The in vitro transcription was then carried out using a RiboMAX Large Scale RNA Production System—T7 (Promega) according to the manufacturer's instructions.

2. 5′-UTR of human rhinovirus 14

The DNA corresponding to the 5′-UTR of human rhinovirus 14 was obtained in the vector pCR2.1 (Stratagene) from Prof. Zeichhardt (Benjamin Franklin university clinic of Berlin Free University). After linearisation with BamHI, an in vitro transcription was carried out as in the case of the VR1 mRNA in order to obtain the target RNA.

Measurement of DNA Enzyme Activities

Enzyme activities were measured in 50 mM Tris-HCl pH 7.5 and 10 mM MgCl₂ at 37° C. In the case of “single turnover” (STO) kinetics, the DNA enzymes were used in a 1 0-fold excess (100 nM RNA substrate; 1 μM DNA enzyme) relative to the substrate. For “multiple turnover” (MTO) experiments, the RNA substrate was used in a 10-fold excess (100 nM RNA substrate; 10 nM DNA enzyme). Determinations were carried out in triplicate in each case.

Effect of Modifications in the Substrate Recognition Arms on the Activity of DNA Enzymes

The activities of the unmodified DNA enzymes DH-5 and DV-1 5 with substrate recognition arms having 9 nucleotides (DH5-9, DV15-9) and 7 nucleotides (DH5-7) were. compared under STO and MTO conditions with the following DNA enzymes having the same base sequence and having chemical modifications in the substrate recognition arms (in the case of the 2′-O-methyl ribose- and LNA-modified species, the modified nucleotides were in each case located at the 5′-end of the first substrate recognition arm (section I) and at the 3′-end of the second substrate recognition arm (section III).):

TABLE 1
DMA enzymes with specific nucleotide modifications
in the substrate recognition arms

| Name | Length of the arms | Modification |
|---|---|---|
| OMe9-4 | 9 | 2′-O-methyl ribose, 4 nucleotides |
| OMe8-4 | 8 | 2′-O-methyl ribose, 4 nucleotides |
| OMe7-3 | 7 | 2′-O-methyl ribose, 3 nucleotides |
| OMe7-4 | 7 | 2′-O-methyl ribose, 4 nucleotides |
| OMe7-5 | 7 | 2′-O-methyl ribose, 5 nucleotides |
| OMe7-6 | 7 | 2′-O-methyl ribose, 6 nucleotides |
| OMe7-7 | 7 | 2′-O-methyl ribose, 7 nucleotides |
| OMe6-5 | 6 | 2′-O-methyl ribose, 5 nucleotides |
| DH5-iT | 9 | inverted thymidine, 1 nucleotide |
| DH5-Thio | 9 | phosphorothioate, 9 nucleotides |
| LH5-9/4 | 9 | LNA ribose, 4 nucleotides |
| LH5-7/3 | 7 | LNA ribose, 4 nucleotides |
| LH5-7/4 | 7 | LNA ribose, 4 nucleotides |

The results of the comparison experiments with the constructs against human rhinovirus 14 (i.e. DH5 constructs) are shown in FIG. 4, the activity of the unmodified DNA enzyme having substrate recognition arms with a length of 9 nucleotides (DH5-9) being set at 1. The activities under MTO conditions, which are critical in particular for in vivo applications, show that DNA enzymes modified in the substrate recognition arms do not exhibit any substantial losses of activity compared with the unmodified comparison constructs (DH5-9 and DH5-7). Moreover, it is to be seen that 2′-O-methyl ribose- and LNA-modified constructs, in particular those in which not all the nucleotides have been modified or in which the length of the substrate recognition arms has been adjusted, exhibit an activity that is in some cases multiplied compared with the unmodified comparison construct. In the case of 2′-O-methyl ribose-modified constructs (3, 4 or 5 modified nucleotides, substrate recognition arms having 7 or 8 nucleotides), activities under MTO conditions that are more than 10 times to more than 20 times greater than those of the unmodified DNA enzymes are obtained.

In the case of the DNA enzyme against VR1 (DV15), the following value of the initial velocity under MTO conditions was obtained for the construct with 5 2′-O-methyl ribonucleotides at the ends of substrate recognition arms having a length of 7 nucleotides, compared with the unmodified construct:

| | |
|---|---|
| DV15 (unmod.): | 0.5 ± 0.1 nM/min |
| DV15-7/5: | 1.7 ± 0.2 nM/min |

Accordingly, the modified construct is more than three times as active against VR1 as the corresponding unmodified DNA enzyme.

### EXAMPLE 2

Dependence of the reaction Velocity on the Melting Point Between DNA Enzyme and Substrate

The modifications 2′-O-methyl and LNA ribonucleotides according to the invention increase the affinity for the substrate (i.e. the melting point of the helices formed between section I or III with the target sequence increases). However, for optimum activity under MTO conditions, efficient product release is also necessary, which is why the affinity for the substrate should not be too high. If, therefore, in the case of the DNA enzymes modified according to the invention according to the above Table 1, the initial velocity under MTO conditions (vᵢₙᵢₜ) is plotted in dependence on the melting temperature with the substrate, a correlation is observed between the melting temperature and the reaction velocity, which can be compared in a first approximation to a Gaussian distribution around an optimum at about 39° C. (FIG. 5).

### EXAMPLE 3

Effect of Modifications in the Core Sequence on the Activity of DNA Enzymes

The 15 nucleotides of the core sequence of DH5 were replaced individually by corresponding 2′-O-methyl ribonucleotides, and the reaction velocity under MTO conditions was measured as indicated in Example 1. It was found that nucleotides 2, 7, 8, 9, 11, 14 and 15 could be replaced by modified nucleotides without any loss of activity (FIG. 6). There was even a gain in activity of at least 20%.

Furthermore, the 6 nucleotides in the case of DH5 were together replaced by 2′-O-methyl ribose-modified nucleotides (construct CM6). The activity was substantially maintained compared with the unmodified construct (FIG. 4; compare CM6 with DH5-9).

In the case of the DNA enzyme against VR1, the above nucleotides of the core sequence could be modified together, the activity compared with the unmodified construct not only being maintained but an increased activity even being observed:

| | |
|---|---|
| DV15(unmod.): | 0.5 ± 0.1 nM/min |
| DV15-CM6: | 0.8 ± 0.2 nM/min |

### EXAMPLE 4

Effect of the combination of Core Sequence and Substrate Recognition Arm Modifications on the Activity of DNA Enzymes

The initial reaction velocity under MTO conditions was also studied in the case of the DNA enzyme against human rhinovirus 14 having substrate recognition arms with a length of 7 nucleotides, in which nucleotides 2, 7, 8, 11, 14 and 15 of the core sequence and in each case 5 nucleotides from the end of the substrate recognition arms were replaced by corresponding 2′-O-methyl ribonucleotides.

The activity of the modified, i.e. completely stabilised, construct compared with the unmodified construct was increased almost 10-fold:

| | | |
|---|---|---|
| DH5-9 (unmod.): | 0.21 ± 0.03 | nM/min |
| DH5-9 (comp. stab.): | 2.0 ± 0.1 | nM/min |

### EXAMPLE 5

### Comparison of the Efficiency of the Inhibition of VR1 Expression by siRNA with Different Antisense Oligonucleotide Constructs

siRNA and Antisense Oligonucleotides

VR1-specific siRNA was prepared by IBA GmbH (Göttingen, Germany) or MWG Biotech AG (Ebersberg, Germany) as a deprotected and desalinated duplex molecule. siRNA according to the invention is directed against the target sequence of type AA(N₁₉)TT. The sequences of the example constructs are shown in Table 2.

TABLE 2
Sequences of siRNA example constructs

| | | |
|---|---|---|
| Construct | Sense strand | Antisense strand |

| | | |
|---|---|---|
| VsiRNA1 | 5′-GCGCAUCUUCUACUUCAACdTdT-3′ | 5′-GUUGAAGUAGAAGAUGCGCdTdT-3′ |
| VsiRNA2 | 5′-GUUCGUGACAAGCAUGUACdTdT-3′ | 5′-GUACAUGCUUGUCACGAACdTdT-3′ |
| VsiRNA3 | 5′-GCAUGUACAACGAGAUCUUdTdT-3′ | 5′-AAGAUCUCGUUGUACAUGCdTdT-3′ |
| VsiRNA4 | 5′-CCGUCAUGACAUGCUUCUCdTdT-3′ | 5′-GAGAAGCAUGUCAUGACGGdTdT-3′ |
| VsiRNA5 | 5′-GAAUAACUCUCUGCCUAUGdTdT-3′ | 5′-CAUAGGCAGAGAGUUAUUCdTdT-3′ |
| VsiRNA6 | 5′-UGUGGGUAUCAUCAACGAGdTdT-3′ | 5′-CUCGUUGAUGAUACCCACAdTdT-3′ |

Unmodified and modified ODN and phosphorothioates were acquired from MWG-Biotech AG (Ebersberg, Germany). 2′-O-Methyl RNA was obtained from IBA GmbH (Göttingen, Germany). LNA (LNA/DNA gapmers) were acquired from Proligo (Boulder, Colo., USA). The sequences of the antisense oligonucleotides were:

V15: 5′-CATGTCATGACGGTTAGG-3′
V30: 5′-ATCTTGTTGACGGTCTCA-3′

The following inverted sequences were used as controls:

V15inv: 5′-GGATTGGCAGTACTGTAC-3′
V30inv: 5′-ACTCTGGCAGTTGTTCTA-3′

With regard to the siRNA, the sense strand thereof served as negative control.

The sequence of LNA/DNA gapmers corresponding to V30 contained eight or ten unmodified DNA monomers in the centre and five or four LNA monomers at each end. The 2′-O-methyl-modified V30 oligonucleotide was likewise synthesised as a gapmer with five 2′-O-methyl ribonucleotides at each end and eight oligodeoxynucleotides in the centre.

Cell Culture and Transfection

COS-7 cells (kidney fibroblasts of the African green ape) were cultured at 37° C. in a moist atmosphere containing 5% CO₂ in DMEM (PM Laboratories, Germany), containing 10% FCS (PM Laboratories, Germany), penicillin (100 μg/ml) and streptomycin (100 μg/ml) (both antibiotics from Invitrogen, Germany). The cells were passaged by dilution (1:10) before reaching confluence in order to keep them in the exponential growth phase. On the day before transfection, the cells were trypsinised, resuspended in medium without antibiotics and transferred in a volume of 500 μl to 24-well plates in a density of 8×10⁴ cells per well. Transfections were carried out with Lipofectamine 2000 (Invitrogen, Germany). A pcDNA3.1/CT-GFP-TOPO plasmid (Invitrogen, Germany), which codes for the VR1-GFP fusion protein, was used. For each transfection, 1 μg of plasmid DNA and the respective amount of antisense oligonucleotide or siRNA were mixed with 50 μl of OPTIMEM (Invitrogen, Germany). In a separate batch, 2.5 μl of Lipofectamine 2000 were added to 50 μl of OPTIMEM for each reaction, and incubation was carried out for 5 minutes at room temperature. The two solutions were mixed and incubated for a further 20 minutes at room temperature for complex formation. The solutions were then added to the cells in the 24-well plate, the final volume being 600 μl. The cells were incubated at 37° C. in the presence of the transfection solution for at least 24 hours.

Fluorescence Microscopy and Immunoblot

The transfection efficiency and the inhibition of the expression of the VR1-GFP fusion protein were analysed by fluorescence microscopy and Western blot. The medium was removed from the cells by suction, 200 μl of phosphate-buffered salt solution (PBS) were added, and fluorescence images of the the living cells were immediately taken using a Leica DM IRB fluorescence microscope.

For Western blot experiments, the cells were immediately lysed in 24-well plates with lysis buffer (125 mM Tris-HCl pH 6.8, 4% SDS, 1.4 M β-mercaptoethanol, 25% glycerol and 0.05% bromophenol blue). The whole of the lysate was boiled for 5 minutes at 95° C., and equal protein amounts were separated in 10% polyacrylamide gels. Transfer of the separated proteins to PVDF membranes (Amersham, Germany) was carried out using a Blot device by the half-dry process (BioRad, Germany). For immune reactions, the membranes were incubated with a GFP antiserum (Invitrogen, Germany) (dilution 1:5000). Secondary antibodies were coupled with alkaline phosphatase (AP) (Chemicon, Germany) and diluted 1:5000. CDP-Star (Roche, Germany) was used as the chemiluminescence substrate. In order to check that equal protein amounts were plotted, the membranes were subjected to a further immune reaction with a monoclonal mouse antibody against actin (Chemicon, Germany).

Quantification of Antisense Effects on Protein Amounts

Protein amounts on Western blots were quantified with the aid of Quantity One software (BioRad, Germany). The antisense effects were calculated by dividing the amount of VR1 protein in the presence of the antisense oligonucleotides or siRNA or the control oligonucleotides. All the values were normalised to the amount of actin as internal standard. The values were matched approximately to a sigmoidal Boltzmann equation using Origin software (Microcal Software, Northampton, Mass,, USA) in order to estimate IC₅₀ values. Means and standard deviations of three independent determinations were calculated.

Comparison of the Inhibition of VR1 Expression by siRNA and Antisense Oligonucleotides

In a mammal culture cell line, the inhibition effectiveness in respect of the expression of VR1 (here: VR1-GFP fusion protein) of VR1-specific siRNA and 18-mer antisense ODN against the target site V30 was compared, the latter being present in the form of a completely phosphorothioate-modified construct, a 2′-O-methyl or a LNA gapmer. The VR1-GFP plasmid was cotransfected together with the different antisense molecules or with the siRNA in the nanomolar concentration range.

All the siRNA species according to the invention led to an antisense effect (inhibition of VR1 expression) of markedly over 50%. Four of the six example constructs used (VsiRNA1, VsiRNA2, VsiRNA3 and VsiRNA5, see Table 2 above) led to an inhibition of VR1 expression of far greater than 80%. The siRNA according to the invention having the most pronounced antisense effect (VsiRNA1) was used for the following comparison experiments.

As the fluorescence microscope images show (FIG. 7A), siRNA inhibits VR1-GFP expression completely at a concentration of only 10 nM. The LNA gapmer leads to a substantial downregulation in VR1-GFP expression only at a concentration of 25 nM. It was possible to observe a partial antisense effect in the case of the phosphorothioate-modified ODN at a higher concentration, while the 2′-O-methyl-modified oligonucleotide did not bring about any suppression of VR1-GFP expression in the observed concentration range.

The antisense and siRNA experiments were analysed more closely by Western blot (FIG. 7B). For comparison purposes, oligonucleotides of identical construction but inverted sequence were used. The Western blot experiments confirm the results obtained by means of fluorescence microscopy. Accordingly, both procedures—fluorescence microscopy and Western blot—gave the same order of the efficiency of blocking of VR1 gene expression for the particular construct: siRNA>LNA gapmer>phosphorothioate ODN>2′-O-methyl-modified oligonucleotide.

Estimation of IC₅₀ Values

In order to quantify the potential of the different strategies—siRNA versus antisense ODN—IC₅₀ values were carried out with the aid of experiments in the suitable concentration range for the siRNA or each antisense oligonucleotide (FIG. 8). The results of the quantitative evaluation (means of determinations in triplicate) are shown in FIG. 9. The IC₅₀ values are summarised in Table 3.

TABLE 3
Estimated IC₅₀ values (individual determinations of three independent
experiments, their means and standard deviations)

| | IC₅₀, 1st exp. | IC₅₀, 2nd exp. | IC₅₀, 3rd exp. | IC_{50, mean} |
|---|---|---|---|---|
| | [nM] | [nM] | [nM] | [nM] |
| Thio | 48.9 | 61.0 | 85.7 | 70 ± 20 |
| LNA | 0.47 | 0.34 | 0.37 | 0.4 ± 0.07 |
| 2′-Ome | 211.4 | 197.5 | 264.8 | 220 ± 10 |
| SiRNA | 0.043 | 0.065 | 0.081 | 0.06 ± 0.02 |

As has already been demonstrated by means of the fluorescence microscopic analysis and the study of the results of the Western blot experiments, the siRNA according to the invention exhibits an extremely high potential in respect of the inhibition of VR1 expression, a significant effect occurring at a concentration of only 0.05 nM and an IC₅₀ value of 0.06 nM being measured. In comparison with phosphorothioate-modified antisense ODN conventionally employed, the siRNA according to the invention therefore proves to be about 1000 times more effective. The LNA gapmer, already optimised in comparison with conventional antisense oligonucleotides, is also markedly inferior to the siRNA according to the invention, with an IC₅₀ value that is 6.5 times higher, while the activity of siRNA is more than 3000 times greater than that of the 2′-O-methyl gapmer.

### EXAMPLE 6

Effectiveness of siRNA Against VR1 in the Treatment of Pain in vivo

Rat Pain Model According to Bennett

The analgesic action of the siRNA of Example 5 according to the invention was studied in vivo in the rat model.

Neuropathic pain occurs inter alia after damage to peripheral or central nerves and can accordingly be induced and observed in animal experiments by targeted lesions of individual nerves. An animal model is nerve lesion according to Bennett (Bennett and Xie (1988) Pain 33: 87-107). In the Bennett model, the sciatic nerve is provided unilaterally with loose ligatures. The development of signs of neuropathic pain is to be observed and can be quantified by means of thermal or mechanical allodynia.

To this end, male Sprague-Dawley rats (Janvier, France) weighing from 140 to 160 grams were first anaesthetised with pentobarbital (50 mg per kg body weight of the rat of Nembutal®, i.p., Sanofi, Wirtschaftsgenossenschaft deutscher Tierärzte eG, Hanover, Germany). Multiple unilateral ligatures were then carried out on the right main sciatic nerve of the rats. To that end, the sciatic nerve was exposed at the level of the middle of the thigh and four loose ligatures (softcat®chrom USP 4/0, metric2, Braun Melsungen, Germany) were tied round the sciatic nerve in such a manner that epineural blood flow was not interrupted. The day of the operation was day 1.

Allodynia was checked from day 2 on a metal plate which was adjusted to a temperature of 4° C. by means of a water bath. The rats were divided into groups of 9 or 10 animals before intravenous administration of the respective solution. To check for allodynia, the rats were placed on the cold metal plate, which was in a plastics cage. Then, over a period of 2 minutes before administration of a solution, the number of times the animals emphatically withdrew the injured paw from the cooled metal plate was counted (preliminary value). The solutions, containing 3.16 μg (5 μl) of siRNA according to the invention in 15 μl of NaCl or 3.16 μg (5 μl) of control RNA (sense strand of siRNA) in 15 μl of NaCl were administered i.t., and after 60 minutes the number of withdrawal reactions was again counted for 2 minutes (test value). The measurements were carried out on four successive days (days 2 to 5). Animals which received pure NaCl solution served as the comparison group in the experiments with both siRNA and control RNA.

The siRNA according to the invention exhibited a potent analgesic action in this pain model, as indicated by the reduction in withdrawal reactions of up to about ⅓ as compared with the NaCl control on days 2 to 4 (FIG. 10A). By comparison, the control RNA was ineffective (FIG. 10B). A single i.t. administration of 1 ng of siRNA leads to a marked and lasting anti-allodynic action in the case of cold allodynia.

### EXAMPLE 7

Kinetic Analysis with Long target RNA

The kinetic experiments with long target RNA were carried out in 50 mM Tris-HCl, 10 mM MgCl₂ and 1 U/μl RNAsin, in order to avoid non-specific RNA degradation. The DNAzymes were denatured for 2 minutes at 65° C. and then cooled to 37° C. The reactions were started by addition of DNAzyme to 100 nM long target solution. The enzyme concentrations for single and multiple turnover experiments were 1 μM and 10 nM. Aliquots were removed after defined intervals during the first 10% of the reaction in the case of multiple turnover conditions and during a prolonged period for single turnover tests. The reaction was stopped by addition of 83 mM EDTA and cooling with ice. The cleavage reactions were analysed by agarose gel electrophoresis and ethidium bromide labelling. The band intensities were quantified using Quantity One software (Bio-Rad, Munich, Germany). The data were analysed further by “fitting” (either linearly to obtain an initial velocity vᵢₙᵢₜ for substrate excess experiments or with a single exponential function to obtain the observed cleavage velocity for enzyme excess experiments and use of Origin (Microcal Software, Northampton, Mass.). The values are means±standard deviation of at least 3 independent experiments.

Stability Assay

Resistance to nucleolytic degradation of various DNAzymes in the cell culture medium was evaluated. DNAzyme (1 μM) was incubated at 37° C. in DMEM (Cytogen, Sinn, Germany) containing 10% FCS (PM Laboratories, Linz, Austria). The samples were removed at defined times between 0 and 72 hours and the continuing reactions were interrupted by the addition of an equal amount of 9 M urea in TBE and subsequent freezing in liquid nitrogen. The oligonucleotides were extracted with phenol and precipitated overnight at −20° C. by addition of sodium acetate, pH 5.2, so that a final concentration of 0.3 M and addition of 2.5 vol. of ethanol was carried out. The precipitate was washed with 70% ethanol and resuspended in a suitable amount of water. After denaturing for 5 minutes at 85° C., degradation products were separated on 20% denaturing polyacrylamide gel. Further analysis was carried out using the Quantity One program (Bio-Rad, Munich, Germany). The half-lives of DNAzyme were obtained by “fitting” the amount of full-length oligonucleotide at different times to a first-order exponential function using Origin (Microcal Software, Northampton, Mass.).

In order to measure the stability of DNAzyme towards endonucleolytic degradation, 2 μM of oligonucleotides were incubated with 0.4 U of S1 endonuclease (Promega, Madison, Wis.) per 100 pmol of DNAzyme in the manufacturer's buffer (50 mM sodium acetate, pH 4.5, 280 mM NaCl, 4.5 mM ZnSO₄). Aliquots were removed after defined times at intervals of from 0 to 180 minutes. The reactions were interrupted by heating at 98° C. for 3 minutes and subsequent freezing in liquid nitrogen. The oligonucleotides were precipitated by ethanol and treated further as described above for the stability test in the cell culture medium. The indicated values are average values±standard deviation of at least 3 independent experiments.

TABLE 4

| | | | T_{M} | k_{obs} | vᵢₙᵢₜ |
|---|---|---|---|---|---|
| DNAzyme | Arm length | Modification | (° C.) | (min⁻¹) | (nM*min⁻¹) |
| DH5-9/0 | 9 | none | 32 | 0.057 ± 0.005 | 0.21 ± 0.03 |
| DH5-7/0 | 7 | none | n.d. (<25) | 0.033 ± 0.002 | n.d. (<0.05) |
| DH5-iT | 9 | inverted T at the 3′-end | 33 | 0.052 ± 0.005 | 0.19 ± 0.01 |
| DH5-Thio | 9 | all-phosphorothioate binding | 27 | 0.009 ± 0.001 | n.d.(<0.05) |
| | | arms | | | |
| LH5-9/4 | 9 | 4 LNA end blocks | 63 | 0.24 ± 0.02 | 0.08 ± 0.02 |
| LH5-7/3 | 7 | 3 LNA end blocks | 48 | 0.45 ± 0.01 | 1.2 ± 0.1 |
| LH5-7/4 | 7 | 4 LNA end blocks | 61 | 0.44 ± 0.05 | 0.45 ± 0.09 |
| DH5-OMe9/4 | 9 | 4 OMe end blocks | 47 | 0.11 ± 0.01 | 1.2 ± 0.1 |
| DH5-OMe8/4 | 8 | 4 OMe end blocks | 44 | 0.29 ± 0.01 | 2.8 ± 0.5 |
| DH5-OMe7/3 | 7 | 3 OMe end blocks | 32 | 0.12 ± 0.03 | 3.8 ± 0.3 |
| DH5-OMe7/4 | 7 | 4 OMe end blocks | 37 | 0.31 ± 0.01 | 4.7 ± 0.4 |
| DH5-OMe7/5 | 7 | 5 OMe end blocks | 39 | 0.5 ± 0.1 | 4.7 ± 0.9 |
| DH5-OMe7/6 | 7 | 6 OMe end blocks | 44 | 0.23 ± 0.06 | 1.9 ± 0.5 |
| DH5-OMe7/7 | 7 | 7 OMe end blocks | 47 | 0.027 ± 0.006 | 0.21 ± 0.02 |
| DH5-OMe6/5 | 6 | 5 OMe end blocks | 26 | 0.17 ± 0.01 | 1.1 ± 0.2 |
| DH5-CM6 | 9 | 6 OMe in the catalytic centre | 31 | 0.06 ± 0.01 | 0.09 ± 0.01 |
| DH5 E | 7 | 5 OMe end blocks; 6 OMe in | 39 | 0.57 ± 0.07 | 2.0 ± 0.2 |
| | | the catalytic centre | | | |
| DV15 9/0 | 9 | none | 37 | 0.9 ± 0.1 | 0.5 ± 0.1 |
| DV15-OMe 7/5 | 7 | 5 OMe end blocks | 40 | 0.83 ± 0.07 | 1.7 ± 0.2 |
| DV15-CM6 | 9 | 6 OMe in the catalytic centre | 36 | 0.43 ± 0.03 | 0.8 ± 0.2 |
| DV15 E | 7 | 5 OMe end blocks; 6 OMe in | 37 | 0.05 ± 0.01 | n.d.(<0.05) |
| | | the catalytic centre | | | |
| DV15 E4 | 7 | 4 OMe end blocks, 6 OMe in | 36 | 0.31 ± 0.08 | 1.3 ± 0.2 |
| | | the catalytic centre | | | |

In Table 4 the studied DNAzymes are shown with their respective arm length and the appropriately conducted modifications. Table 4 also contains the melting point (Tₘ(° C.)) and the observed cleavage rates (min⁻¹) (k_{obs}) in single turnover experiments and the initial velocities (vᵢₙᵢₜ) in multiple turnover experiments (in each case with long target RNA). In the names of the DNAzymes, the number of nucleotides in each binding arm is in each case indicated before the slash. The figure after the slash relates to the number of modified nucleotides in each binding arm. The abbreviation OMe stands for the 2′-O-methyl modification. The abbreviation iT stands for 3′-inverted thymidine. Thio means that the binding arms all contain phosphorothioates. L stands for the LNA modification. Tₘ indicates the melting temperatures of the target molecule/enzyme duplexes.

TABLE 5

| | | t_{1/2} | t_{1/2} | Performance |
|---|---|---|---|---|
| | Vᵢₙᵢₜ | Medium | S1-Nucl. | index |
| DH5-9/0 | 1 | 1 | 1 | 1 |
| DH5-Thio | <0.23 | 11.5 | 1.6 | <4 |
| DH5-iT | 0.9 | 11.5 | 1 | 10 |
| DH5-OMe7/5 | 22.4 | 3.5 | 1 | 78 |
| LH5-7/3 | 5.7 | 9.5 | 1.5 | 81 |
| DH5 E | 9.7 | 12.5 | 2.1 | 255 |

Table 5 contains a summary of the results for various modified DNAzymes for comparison. The initial velocity under the different turnover conditions, namely in the cell culture medium, and the stability towards endonuclease S1 are given. All values are normalised to unmodified DNAzyme. An index of the overall result of the modified DNAzymes from three values is indicated in the last column. With regard to the abbreviations used, reference is made to Table 4 and the associated explanations.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations falling within the scope of the appended claims and equivalents thereof.

What is claimed is:

1. A DNA enzyme of type 10-23, comprising: from the 5′ to the 3′ end, a first substrate recognition arm (section 1), a catalytic core sequence (section II) and a second substrate recognition arm (section III), wherein one or more of the nucleotides 2, 7, 8, 11, 14 and 15 of section 11 are modified.

2. A DNA enzyme according to claim 1, wherein all the nucleotides 2, 7, 8, 11, 14 and 15 of section 11 are modified.

3. A DNA enzyme according to claim 1, wherein one or more of the nucleotides of section I or of section III are modified.

4. A DNA enzyme according to claim 3, wherein from 3 to 5 nucleotides of section I or of section III have been modified.

5. A DNA enzyme according to claim 4, wherein the modified nucleotides are located at the 5′-end of section I and/or at the 3′-end of section III.

6. A DNA enzyme according to claim 4, wherein the modified nucleotides of section I or of section III are 2′-O-methyl ribonucleotides or LNA ribonucleotides.

7. A DNA enzyme according to claim 1, wherein the one or more modified nucleotides are selected from the group consisting of phosphorothioate nucleotides, inverted thymidine, 2′-O-methyl ribonucleotides and LNA ribonucleotides.

8. A DNA enzyme according to claim 7, wherein from 3 to 5 nucleotides of section I or of section III have been modified.

9. A DNA enzyme according to claim 8, wherein the modified nucleotides are located at the 5′-end of section I and/or at the 3′-end of section III.

10. A DNA enzyme according to claim 8, wherein the modified nucleotides of section I or of section III are 2′-O-methyl ribonucleotides or LNA ribonucleotides.

11. A DNA enzyme according to claim 1, wherein either of section I or section III comprises no more than 8 nucleotides.

12. A DNA enzyme according to claim 11, wherein section I or section III comprises 7 nucleotides.

13. A DNA enzyme according to claim 3, wherein all the nucleotides of section I or of section III are phosphorothioate nucleotides or 2′-O-methyl ribonucleotides.

14. A DNA enzyme according to claim 3, wherein the melting temperature of the double strands formed between sections I and III and the target molecule is from about 33 to about 42° C.

15. A DNA enzyme according to claim 1, wherein section II exhibits the following consensus sequence from 5′ to 3′:
GGMTMGH(N)DNNNMGD
where M=A or C;
H=A, C, or T;
D=G, A or T; and
N=any base.

16. A DNA enzyme according to claim 1 which is directed against the mRNA of the vanilloid receptor 1.

17. A DNA enzyme according to claim 16, wherein sections I and III comprise, from 5′ to 3′, a sequence selected from the respective group consisting of:

| | |
|---|---|
| Section I | Section III |
| | |
|---|---|
| GTCATGA | GGTTAGG |
| TGTCATGA | GGTTAGGG |
| ATGTCATGA | GGTTAGGGG |
| GTCGTGG | GATTAGG |
| TGTCGTGG | GATTAGG |
| ATGTCGTGG | GATTAGG |
| TTGTTGA | GGTCTCA |
| CTTGTTGA | GGTCTCAC |
| TCTTGTTGA | GGTCTCACC |
| TTGTTGA | AGTCTCA |
| CTTGTTGA | AGTCTCAN |
| TCTTGTTGA | AGTCTCANN |
| GGCCTGA | CTCAGGG |
| CGGCCTGA | CTCAGGGA |
| TCGGCCTGA | CTCAGGGAG |
| TGCTTGA | CGCAGGG |
| CTGCTTGA | CGCAGGGN |
| TCTGCTTGA | CGCAGGGNN |
| GTGTGGA | TCCATAG |
| GGTGTGGA | TCCATAGG |
| TGGTGTGGA | TCCATAGGC |
| ACGTGGA | TCAGACG |
| GACGTGGA | TCAGACGN |
| CGACGTGGA | TCAGACGNN |
| GTGGGGA | TCAGACT |
| GGTGGGGA | TCAGACTC |
| GGGTGGGGA | TCAGACTCC |
| GTGGGTC | GCAGCAG |
| AGTGGGTC | GCAGCAG |
| GAGTGGGTC | GCAGCAG |
| CGCTTGA | AAATCTG |
| GCGCTTGA | AAATCTGT |
| TGCGCTTGA | AAATCTGTC |
| CGCTTGA | GAATCTG |
| GCGCTTGA | GAATCTGN |
| TGCGCTTGA | GAATCTGNN |
| CTCCAGA | ATGTGGA |
| GCTCCAGA | ATGTGGAA |
| AGCTCCAGA | ATGTGGAAT |
| CTCCAGG | AGGTGGA |
| GCTCCAGG | AGGTGGA |
| AGCTCCAGG | AGGTGGA |
| GGTACGA | TCCTGGT |
| GGGTACGA | TCCTGGTA |
| CGGGTACGA | TCCTGGTAG |
| GGTGCGG | TCTTGGC |
| GGGTGCGG | TCTTGGC |
| CGGGTGCGG | TCTTGGC |

where N=any base, or a sequence differing therefrom by a nucleotide, with the proviso that the nucleotide differing from the indicated sequences is not located at one of the last three positions of section I nor at one of the first three positions of section III.

18. An siRNA directed against a target sequence of VR1-mRNA, which siRNA corresponds to the structure 5′-AA(N₁₉)TT-3′.

19. An siRNA according to claim 18, wherein the target sequence is a sequence selected from the group consisting of
5′-AAGCGCAUCUUCUACUUCAACTT-3′,
5′-AAGUUCGUGACAAGCAUGUACTT-3′,
5′-AAGCAUGUACAACGAGAUCUUTT-3′,
5′-AACCGUCAUGACAUGCUUCUCTT-3′,
5′-AAGAAUAACUCUCUGCCUAUGTT-3′
and
5′-AAUGUGGGUAUCAUCAACGAGTT-3′.

20. An siRNA according to claim 19, wherein said siRNA is selected from the group of duplex molecules consisting of:
Sense Strand/Antisense Strand
5′-GCGCAUCUUCUACUUCAACdTdT-3′/5′-GUUGAAGUAGAAGAUGCGCdTdT-3′,
5′-GUUCGUGACAAGCAUGUACdTdT-3′/5′-GUACAUGCUUGUCACGAACdTdT-3′,
5′-GCAUGUACAACGAGAUCUUdTdT-3′/5′-AAGAUCUCGUUGUACAUGCdTdT-3′,
5′-CCGUCAUGACAUGCUUCUCdTdT-3′/5′-GAGAAGCAUGUCAUGACGGdTdT-3′,
5′-GAAUAACUCUCUGCCUAUGdTdT-3′/5′-CAUAGGCAGAGAGUUAUUCdTdT-3′
and
5′-UGUGGGUAUCAUCAACGAGdTdT-3′/5′-CUCGUUGAUGAUACCCACAdTdT-3′.

21. A host cell containing at least one DNA enzyme according to claim 1 or an siRNA, wherein said siRNA corresponds to the structure 5′-AA(N₁₉)TT-3′, wherein the host cell is not a human germ cell or a human embryonal stem cell.

22. A host cell according to claim 21, wherein said cell is a mammalian cell.

23. A host cell according to claim 22, wherein said cell is a human cell.

24. A process for downregulating the expression of a gene comprising:
introducing at least one DNA enzyme according to claim 1 into a cell expressing the gene.

25. A process according to claim 24, wherein the gene is the VR1 gene and said at least one DNA enzyme is directed against the mRNA of the vanilloid receptor 1.

26. A process for downregulating the expression of the VR1 gene comprising:
introducing at least one siRNA according to claim 18 into a cell expressing the VR1 gene.

27. A pharmaceutical formulation comprising, as an active ingredient, at least one DNA enzyme according to claim 1 and a pharmaceutically acceptable carrier or adjuvant.

28. A pharmaceutical formulation comprising, as an active ingredient, at least one siRNA according to claim 18 and a pharmaceutically acceptable carrier or adjuvant.

29. A pharmaceutical formulation comprising, as an active ingredient, at least one host cell according to claim 21 and a pharmaceutically acceptable carrier or adjuvant.

30. A pharmaceutical formulation comprising, as an active ingredient, at least one DNA enzyme according to claim 16 and a pharmaceutically acceptable carrier or adjuvant.

31. A pharmaceutical formulation comprising, as an active ingredient, at least one DNA enzyme according to claim 17 and a pharmaceutically acceptable carrier or adjuvant.

32. A method of alleviating pain in a mammal, said method comprising administering to said mammal an effective pain alleviating amount of a DNA enzyme according to claim 16.

33. The method of claim 32, wherein said pain is chronic pain, tactile allodynia, thermally initiated pain or inflammatory pain.

34. A method of alleviating pain in a mammal, said method comprising administering to said mammal an effective pain alleviating amount of an siRNA according to claim 18.

35. The method of claim 34, wherein said pain is chronic pain, tactile allodynia, thermally initiated pain or inflammatory pain.

36. A method of alleviating pain in a mammal, said method comprising administering to said mammal an effective pain alleviating amount of an host cell according to claim 21.

37. The method of claim 36, wherein said pain is chronic pain, tactile allodynia, thermally initiated pain or inflammatory pain.

38. A method of treating or inhibiting a condition selected from the group consisting of neurogenic bladder symptoms, urinary incontinence, VR1-associated sensitivity disorders, VR1 -associated inflammations and VR1-associated tumors comprising administering a pharmaceutically effective amount of a DNA enzyme according to claim 16.

39. A method of treating or inhibiting a condition selected from the group consisting of neurogenic bladder symptoms, urinary incontinence, VR1-associated sensitivity disorders, VR1 -associated inflammations and VR1-associated tumors comprising administering a pharmaceutically effective amount of an siRNA according to claim 18.

40. A method of treating or inhibiting a condition selected from the group consisting of neurogenic bladder symptoms, urinary incontinence, VR1-associated sensitivity disorders, VR1-associated inflammations and VR1-associated tumors comprising administering a pharmaceutically effective amount of a host cell according to any one of claim 21.
